(19) United States
(12) Patent Application Publication
(10) Pub. No.: US 2025/0316284 A1
(43) Pub. Date: Oct. 9, 2025

(54) VOICE FEATURE CALCULATION METHOD, VOICE FEATURE CALCULATION DEVICE, AND ORAL FUNCTION EVALUATION DEVICE

(71) Applicant: Panasonic Intellectual Property Management Co., Ltd., Osaka (JP)

(72) Inventors: Masashi ISHIMARU, Osaka (JP); Junko NAKAJIMA, Osaka (JP); Hiroki KAMEI, Osaka (JP); Yaming ZHANG, Hyogo (JP); Jakusei KIYOSAKI, Hyogo (JP); Yoshihiro MATSUMURA, Osaka (JP)

(21) Appl. No.: 18/866,342

(22) PCT Filed: Apr. 12, 2023
(86) PCT No.: PCT/JP2023/014842

§ 371 Date: Nov. 15, 2024

(30) Foreign Application Priority Data
May 25, 2022 (JP) 2022-085002

(51) Int. Cl. G10L 25/66; G10L 21/0332; G10L 25/21

(52) U.S. Cl. CPC G10L 25/66; G10L 21/0332; G10L 25/21

(57) ABSTRACT

A voice feature calculation method, performed by a computer, for calculating one or more features of a voice of an evaluatee from a voice uttered by the evaluatee, the voice feature calculation method including: obtaining voice data obtained by collecting a voice uttered by the evaluatee; adjusting a sound pressure of the voice data obtained, based on a first average intensity of a sound that is included in the voice data obtained and is collected in a period in which the evaluatee does not utter a voice; and calculating, from the voice data resulting from the adjusting of the sound pressure, the one or more features including at least a feature related to a sound pressure.

### TECHNICAL FIELD

The present invention relates to a voice feature calculation method and a voice feature calculation device for calculating a voice feature of an evaluatee, and an oral function evaluation device that uses the voice feature calculation device.

### BACKGROUND ART

A method for evaluating the eating and swallowing function of an evaluatee by obtaining a pharynx movement feature as an eating and swallowing function evaluation indicator (marker) from an appliance which is put on the neck of the evaluatee to evaluate the eating and swallowing function is disclosed (e.g., see Patent Literature (PTL) 1).

### CITATION LIST

### Patent Literature

[PTL 1] Japanese Unexamined Patent Application Publication No. 2017-23676

### SUMMARY OF INVENTION

### Technical Problem

However, the method disclosed in PTL 1 requires an evaluatee to put on the appliance to evaluate oral function such as eating and swallowing function. This may cause discomfort to the evaluatee and impose a burden on the evaluatee. Oral function can be evaluated also by visual inspection, interview, palpation, or the like by a specialist such as a dentist, a dental hygienist, a speech pathologist, or a physician. However, deterioration in the oral function of an elderly person may be overlooked, being regarded as a natural symptom of an elderly person, although the elderly person chokes all the time or spills food because of an influence of aging. Overlooking deterioration in the oral function brings about, for example, undernutrition resulting from a decrease in an amount of food intake, and the undernutrition brings about a decrease in immune strength. In addition, deterioration in the oral function tends to cause aspiration, and as a result, the aspiration and the decrease in immune strength bring about a vicious circle that leads to a risk of aspiration pneumonia.

Even without use of such a method, oral function of an evaluatee can be evaluated from a voice uttered by the evaluatee; however, the accuracy of calculation of a feature of a voice used in the evaluation and so on has been unsatisfactory.

In view of the above, it is an object of the present invention to provide a voice feature calculation method and so on capable of calculating a feature of a voice more appropriately from a voice of an evaluatee.

### Solution to Problem

A voice feature calculation method according to an aspect of the present invention is a voice feature calculation method, performed by a computer, for calculating one or more features of a voice of an evaluatee from a voice uttered by the evaluatee, the voice feature calculation method including: obtaining voice data obtained by collecting a voice uttered by the evaluatee; adjusting a sound pressure of the voice data obtained, based on a first average intensity of a sound that is included in the voice data obtained and is collected in a period in which the evaluatee does not utter a voice; and calculating, from the voice data resulting from the adjusting of the sound pressure, the one or more features including at least a feature related to a sound pressure.

Also, a voice feature calculation device according to an aspect of the present invention is a voice feature calculation device that calculates one or more features of a voice of an evaluatee from a voice uttered by the evaluatee, the voice feature calculation device including: an obtainer that obtains voice data obtained by collecting a voice uttered by the evaluatee; a sound pressure adjuster that adjusts a sound pressure of the voice data obtained, based on a first average intensity of a sound that is included in the voice data and is collected in a period in which the evaluatee does not utter a voice; and an extractor that calculates the one or more features including at least a feature related to a sound pressure, by extracting the one or more features including at least the feature related to a sound pressure, from the voice data resulting from the adjustment of the sound pressure.

Also, an oral function evaluation device according to an aspect of the present invention includes: the voice feature calculation device described above; a calculator that calculates an estimate value of oral function of the evaluatee, based on: an estimating equation including the feature related to a sound pressure among the one or more features extracted from the voice data; and the one or more features extracted from the voice data resulting from the adjustment of the sound pressure; and an evaluator that evaluates a deterioration state of the oral function of the evaluatee by assessing the estimate value using an oral function evaluation indicator.

### Advantageous Effects of Invention

With a voice feature calculation method and so on according to the present invention, it is possible to calculate a feature of a voice more appropriately from a voice of an evaluatee.

### BRIEF DESCRIPTION OF DRAWINGS

FIG. 1 is a diagram illustrating a configuration of an oral function evaluation system according to an embodiment.

FIG. 2 is a block diagram illustrating a characteristic functional configuration of the oral function evaluation system according to the embodiment.

FIG. 3A is a flowchart illustrating a processing procedure for evaluating oral function of an evaluatee using an oral function evaluation method according to the embodiment.

FIG. 3B is a flowchart illustrating a processing procedure related to voice data to be used in the oral function evaluation method according to the embodiment.

FIG. 3C is a diagram illustrating an example of information output in the oral function evaluation method according to the embodiment.

FIG. 3D is a flowchart illustrating a processing procedure related to voice data to be used in the oral function evaluation method according to another example of the embodiment.

FIG. 3E is a first graph for describing adjustment of sound pressure of voice data according to the embodiment.

FIG. 3F is a second graph for describing adjustment of sound pressure of voice data according to the embodiment.

FIG. 3G is a third graph for describing adjustment of sound pressure of voice data according to the embodiment.

FIG. 3H shows graphs each illustrating a relationship between adjustment of sound pressure in the oral function evaluation method according to the embodiment and accuracy (estimation precision).

FIG. 4 is a diagram illustrating an outline of a method for obtaining a voice of an evaluatee using the oral function evaluation method according to the embodiment.

FIG. 5A is a graph illustrating an example of voice data indicating a voice of an evaluatee uttering “e o kaku koto ni kimeta yo.”

FIG. 5B is a graph illustrating an example of changes in formant frequencies of a voice of an evaluatee uttering “e o kaku koto ni kimeta yo.”

FIG. 6 is a graph illustrating an example of voice data indicating a voice of an evaluatee repeatedly uttering “karakarakara. . . . ”

FIG. 7 is a graph illustrating an example of voice data indicating a voice of an evaluatee uttering “ittai.”

FIG. 8 is a table showing an example of syllables and fixed sentences in Japanese and syllables and fixed sentences in Chinese that are similar in tongue movement or degree of mouth opening and closing when pronounced.

FIG. 9A is a diagram illustrating international phonetic alphabet symbols of vowels.

FIG. 9B is a table illustrating international phonetic alphabet symbols of consonants.

FIG. 10A is a graph illustrating an example of voice data indicating a voice of an evaluatee uttering “gao dao wu da ka ji ke da yi wu zhe.”

FIG. 10B is a graph illustrating an example of changes in formant frequencies of a voice of an evaluatee uttering “gao dao wu da ka ji ke da yi wu zhe.”

FIG. 11 is a diagram illustrating an example of oral function evaluation indicators.

FIG. 12 is a table illustrating an example of evaluation results on elements of oral function.

FIG. 13 is a chart illustrating an example of evaluation results on elements of oral function.

FIG. 14 is an example of predetermined data that is used when providing a suggestion regarding oral function.

### DESCRIPTION OF EMBODIMENTS

Hereinafter, embodiments will be described with reference to the drawings. It should be noted that the following embodiments each illustrate a general or specific example. The numerical values, shapes, materials, constituent elements, the arrangement and connection of the constituent elements, steps, the processing order of the steps etc. illustrated in the following embodiments are mere examples, and are not intended to limit the present invention. Among the constituent elements in the following embodiments, those not recited in any of the independent claims representing the most generic concepts will be described as optional constituent elements.

It should be noted that the drawings are represented schematically and are not necessarily precise illustrations. Furthermore, in the drawings, constituent elements that are substantially the same are given the same reference signs, and redundant descriptions will be omitted or simplified.

### Embodiment

### [Elements of Oral Function]

The present invention relates to, for example, a method for evaluating deterioration of oral function, and oral function includes various elements.

For example, elements of oral function include tongue fur adhesion, oral mucous wetness, occlusal force, tongue pressure, cheek pressure, the remaining number of teeth, swallowing function, mastication function, and so on. The following briefly describes tongue fur adhesion, oral mucous wetness, occlusal force, tongue pressure, and mastication function.

The tongue fur adhesion indicates how much bacteria or food is deposited on the tongue. No tongue fur or thin tongue fur shows that there is an environment of mechanical abrasion (food intake, etc.), cleaning action by saliva is present, or swallowing movement (tongue movement) is normal. In contrast, thick tongue fur shows poor tongue movement and a difficulty in taking food, which may bring about malnutrition or poor muscle strength. The oral mucous wetness is a degree of how dry the tongue is, and when the tongue is dry, movement for speech is inhibited. Food is chewed after being taken into the oral cavity, and the food only chewed is difficult to swallow. Thus, to make it easy to swallow chewed food, saliva exercises a function of gathering the chewed food. However, when the oral cavity is dry, it is difficult to form a bolus (chewed food gathered). The occlusal force is the force for biting hard things and is the strength of jaw muscles. The tongue pressure is an indicator that expresses the force of the tongue pressing the palate. When the tongue pressure is weakened, it may be difficult to make movement of swallowing. Furthermore, when the tongue pressure is weakened, the speed of moving the tongue may decrease, and the speech rate may decrease. The mastication function is comprehensive function of the oral cavity.

According to the present invention, it is possible to evaluate a deterioration state of oral function (e.g., a deterioration state of an element of oral function) of an evaluatee from a voice uttered by the evaluatee. This is because a voice uttered by an evaluatee whose oral function is deteriorating has a specific feature, and by extracting the specific feature as a prosody feature, oral function of the evaluatee can be evaluated. The present invention is implemented by an oral function evaluation method, a program that causes a computer or the like to perform the method, an oral function evaluation device that is an example of the computer, and an oral function evaluation system that includes the oral function evaluation device. Hereinafter, the oral function evaluation method and the like will be described along with the oral function evaluation system.

### [Configuration of Oral Function Evaluation System]

A configuration of oral function evaluation system 200 according to an embodiment will be described.

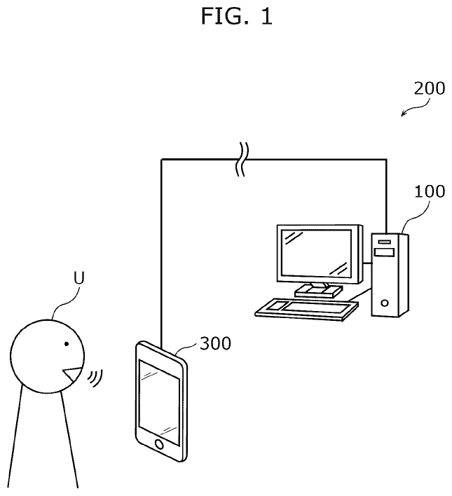

FIG. 1 is a diagram illustrating a configuration of oral function evaluation system 200 according to the embodiment.

Oral function evaluation system 200 is a system for evaluating oral function of evaluatee U by analyzing a voice of evaluatee U. As illustrated in FIG. 1, oral function evaluation system 200 includes oral function evaluation device 100 and mobile terminal 300 (an example of a terminal).

Oral function evaluation device 100 is a device that obtains voice data indicating a voice uttered by evaluatee U through mobile terminal 300 and evaluates oral function of evaluatee U from the voice data obtained.

Mobile terminal 300 is a sound collection device that collects in a contactless manner a voice of evaluatee U uttering a syllable or a fixed sentence that includes (i) two or more morae including a change in a first formant frequency or a change in a second formant frequency or (ii) at least one of a flap, a plosive, a voiceless sound, a double consonant, or a fricative, and outputs voice data indicating the collected voice to oral function evaluation device 100. For example, mobile terminal 300 is a smartphone or a tablet computer including a microphone. It should be noted that mobile terminal 300 is not limited to a smartphone, a tablet computer, or the like so long as it is a device having a sound collecting function. For example, mobile terminal 300 may be a laptop computer. Oral function evaluation system 200 may include a sound collection device (a microphone) instead of mobile terminal 300. Oral function evaluation system 200 may include an input interface for obtaining personal information on evaluatee U. The input interface is not particularly limited so long as it is an input interface having an input function, such as a keyboard or a touch panel. Oral function evaluation system 200 may set the volume of the microphone.

Mobile terminal 300 may be a display device that includes a display and displays, for example, an image based on image data output from oral function evaluation device 100. That is to say, mobile terminal 300 is an example of a presentation device that presents, in the form of an image, information output from oral function evaluation device 100. It should be noted that the display device need not be mobile terminal 300 and may be a monitor device that includes a liquid crystal panel, an organic EL panel, or the like. In other words, although mobile terminal 300 serves as both a sound collection device and a display device in the present embodiment, the sound collection device (microphone), the input interface, and the display device may be provided separately.

It suffices so long as oral function evaluation device 100 and mobile terminal 300 are capable of transmitting and receiving, for example, image data for displaying an image indicating an evaluation result that will be described later or voice data. Thus, oral function evaluation device 100 and mobile terminal 300 may be connected in a wired manner or may be connected in a wireless manner.

Oral function evaluation device 100 analyzes a voice of evaluatee U based on voice data collected by mobile terminal 300, evaluates oral function of evaluatee U from a result of the analysis, and outputs an evaluation result. For example, oral function evaluation device 100 outputs, to mobile terminal 300, image data for displaying an image indicating the evaluation result or data for providing a suggestion to evaluatee U regarding oral function and generated based on the evaluation result. With this configuration, oral function evaluation device 100 can notify evaluatee U of a level of oral function and a suggestion for preventing deterioration of oral function, for example. Thus, evaluatee U can prevent deterioration of oral function or improve oral function, for example.

It should be noted that although oral function evaluation device 100 is, for example, a personal computer, it may be a server device. Further, oral function evaluation device 100 may be mobile terminal 300. That is to say, mobile terminal 300 may have the function of oral function evaluation device 100 described below.

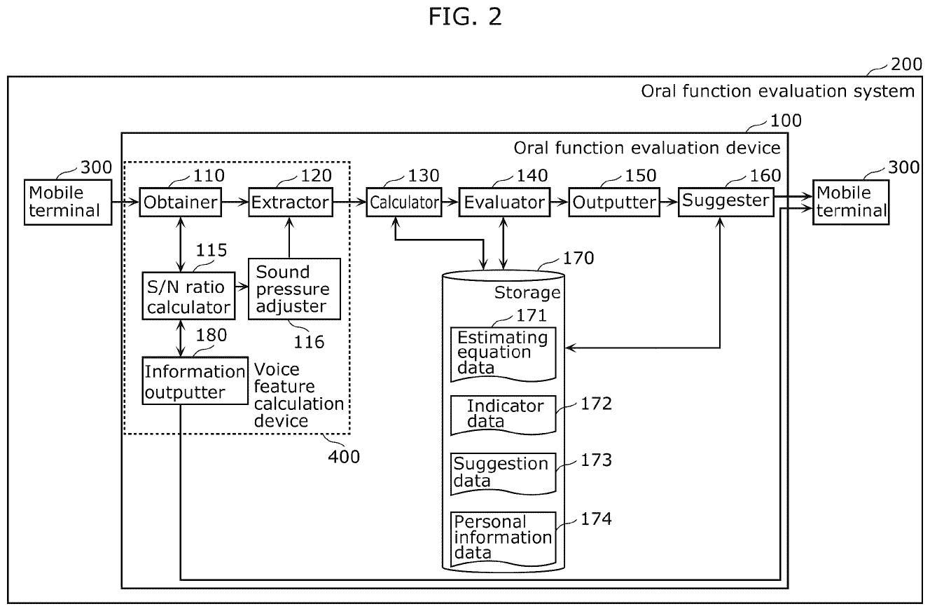

FIG. 2 is a block diagram illustrating a characteristic functional configuration of oral function evaluation system 200 according to the embodiment. Oral function evaluation device 100 includes voice feature calculation device 400, calculator 130, evaluator 140, outputter 150, suggester 160, and storage 170.

Voice feature calculation device 400 is a device that calculates a feature (prosody feature) of a voice of evaluatee U by extracting the feature. Specifically, voice feature calculation device 400 includes obtainer 110, S/N ratio calculator 115, sound pressure adjuster 116, extractor 120, and information outputter 180. It should be noted that although the example given here is a configuration in which voice feature calculation device 400 is included inside oral function evaluation device 100, voice feature calculation device 400 may be provided separately from oral function evaluation device 100. In that case, oral function evaluation device 100 may include, separately from obtainer 110 of voice feature calculation device 400, an obtainer that obtains voice data and personal information, for example.

Obtainer 110 obtains voice data obtained by mobile terminal 300 collecting in a contactless manner a voice uttered by evaluatee U. The voice is a voice of evaluatee U uttering a syllable or a fixed sentence that includes two or more morae including a change in the first formant frequency or a change in the second formant frequency. Alternatively, the voice is a voice of evaluatee U uttering a syllable or a fixed sentence that includes at least one of a flap, a plosive, a voiceless sound, a double consonant, or a fricative. However, in some situations which will be described later, the voice may be a voice of evaluatee U uttering an arbitrary sentence. Obtainer 110 may further obtain personal information on evaluatee U. For example, the personal information is information input to mobile terminal 300 and includes age, weight, height, sex, body mass index (BMI), dental information (e.g., the number of teeth, whether a denture is used, occlusal support location, the number of functional teeth, and the remaining number of teeth), serum albumin level, or eating rate. It should be noted that the personal information may be obtained through a swallowing screening tool called the eating assessment tool-10 (EAT-10), Seirei dysphagia screening questionnaire, interview, Barthel Index, Kihon Checklist, or the like. Obtainer 110 is, for example, a communication interface that performs wired communication or wireless communication.

S/N ratio calculator 115 is a processing unit that calculates a signal-to-noise (S/N) ratio of the voice data obtained. The S/N ratio of the voice data is a ratio of a second average intensity of a sound that is included in the voice data obtained and is collected in a period in which evaluatee U utters a voice to a first average intensity of a sound that is included in the voice data obtained and is collected in a period in which evaluatee U does not utter a voice (a period in which only background noise is collected; hereinafter also referred to as a background noise period). Therefore, S/N ratio calculator 115 is configured capable of calculating the first average intensity by extracting, from the voice data, a sound corresponding to the period in which evaluatee U does not utter a voice and calculating the second average intensity by extracting, from the voice data, a sound corresponding to the period in which evaluatee U utters a voice. Specifically, S/N ratio calculator 115 is implemented by a processor, a microcomputer, or a dedicated circuit.

Sound pressure adjuster 116 is a processing unit that, when the S/N ratio of the voice data obtained indicates a situation unsuitable for evaluation of oral function, performs sound pressure adjustment processing on the voice data to generate adjusted voice data suitable for evaluation of oral function, and outputs the adjusted voice data. The adjustment of the sound pressure of the voice data performed by sound pressure adjuster 116 will be described later. Specifically, sound pressure adjuster 116 is implemented by a processor, a microcomputer, or a dedicated circuit.

Extractor 120 is a processing unit that analyzes the voice data of evaluatee U obtained by obtainer 110 or the voice data resulting from the sound pressure adjustment performed by sound pressure adjuster 116. Specifically, extractor 120 is implemented by a processor, a microcomputer, or a dedicated circuit.

Extractor 120 calculates one or more prosody features by extracting the one or more prosody feature from the voice data obtained by obtainer 110 or the voice data output by sound pressure adjuster 116. A prosody feature is a numerical value indicating a feature of a voice of evaluatee U extracted from voice data used by evaluator 140 to evaluate oral function of evaluatee U. The one or more prosody features include a feature related to a sound pressure including at least one of a sound pressure difference or a change over time in a sound pressure difference. Other than that, the one or more prosody features may include at least one of the speech rate, the first formant frequency, the second formant frequency, an amount of change in the first formant frequency, an amount of change in the second formant frequency, a change over time in the first formant frequency, a change over time in the second formant frequency, a time length with mouth opened, a time length with mouth closed, or a time length of a plosive.

Information outputter 180 is a processing unit that outputs information for increasing the S/N ratio. When the calculated S/N ratio does not meet a certain criterion, information outputter 180 generates and outputs information indicating an instruction to improve the environment in which a voice uttered by evaluatee U is collected. Specifically, information outputter 180 is implemented by a processor, a microcomputer, or a dedicated circuit.

Calculator 130 calculates an estimate value of oral function of evaluatee U, based on the one or more prosody features extracted by extractor 120 and an estimating equation that is set in advance. Specifically, calculator 130 is implemented by a processor, a microcomputer, or a dedicated circuit.

Evaluator 140 evaluates a deterioration state of oral function of evaluatee U by assessing, using an oral function evaluation indicator, the estimate value calculated by calculator 130. Indicator data 172 indicating the oral function evaluation indicator is stored in storage 170. Specifically, evaluator 140 is implemented by a processor, a microcomputer, or a dedicated circuit.

Outputter 150 outputs the estimate value calculated by calculator 130 to suggester 160. Outputter 150 may output an evaluation result on oral function of evaluatee U evaluated by evaluator 140 to mobile terminal 300, for example. Specifically, outputter 150 is implemented by a processor, a microcomputer, or a dedicated circuit, and a communication interface that performs wired communication or wireless communication.

Suggester 160 provides a suggestion regarding oral function of evaluatee U by checking the estimate value calculated by calculator 130 against predetermined data. Suggestion data 173, which is the predetermined data, is stored in storage 170. Suggester 160 may provide a suggestion regarding oral function to evaluatee U by checking, against suggestion data 173, the personal information obtained by obtainer 110. Suggester 160 outputs the suggestion to mobile terminal 300. Suggester 160 is implemented by, for example, a processor, a microcomputer, or a dedicated circuit, and a communication interface that performs wired communication or wireless communication.

Storage 170 is a storage device in which the following data are stored: estimating equation data 171 indicating an oral function estimating equation calculated based on a plurality of training data items; indicator data 172 indicating the oral function evaluation indicator used for assessing the estimate value of oral function of evaluatee U; suggestion data 173 indicating a relationship between the estimate value of oral function and suggestion details; and personal information data 174 indicating the above-described personal information on evaluatee U. Estimating equation data 171 is referred to by calculator 130 when calculating an estimate value of oral function of evaluatee U. Indicator data 172 is referred to by evaluator 140 when evaluating a deterioration state of oral function of evaluatee U. Suggestion data 173 is referred to by suggester 160 when providing a suggestion regarding oral function to evaluatee U. Personal information data 174 is, for example, data obtained via obtainer 110. It should be noted that personal information data 174 may be stored in storage 170 in advance. Storage 170 is implemented by, for example, read-only memory (ROM), random-access memory (RAM), semiconductor memory, hard disk drive (HDD), or the like.

Storage 170 may also store: a program executed by a computer to implement each functional unit of voice feature calculation device 400, calculator 130, evaluator 140, outputter 150, and suggester 160; image data indicating an evaluation result on oral function of evaluatee U and used when the evaluation result is output; and data such as an image, video, voice, or text indicating details of a suggestion. Storage 170 may store an instruction image that will be described later.

Although not illustrated, oral function evaluation device 100 may include an instructor that instructs evaluatee U to utter a syllable or a fixed sentence that includes (i) two or more morae including a change in the first formant frequency or a change in the second formant frequency or (ii) at least one of a flap, a plosive, a voiceless sound, a double consonant, or a fricative. Specifically, the instructor obtains image data on an instruction image or voice data on an instruction voice that is stored in storage 170 and that instructs evaluatee U to utter the syllable or the fixed sentence, and the instructor outputs the image data or the voice data to mobile terminal 300.

### [Processing Procedure of Oral Function Evaluation Method]

Now, a specific processing procedure of an oral function evaluation method executed by oral function evaluation device 100 will be described.

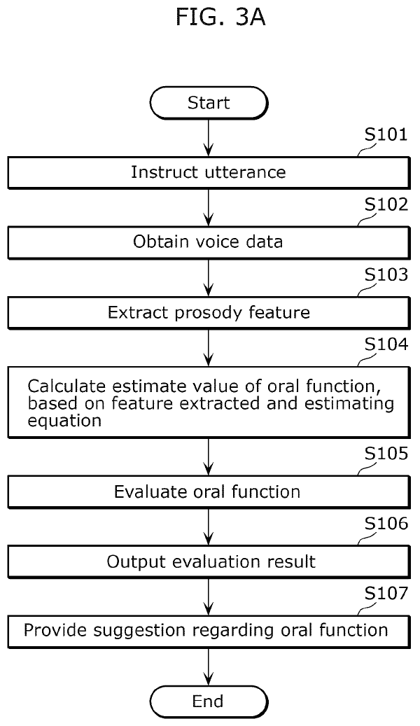

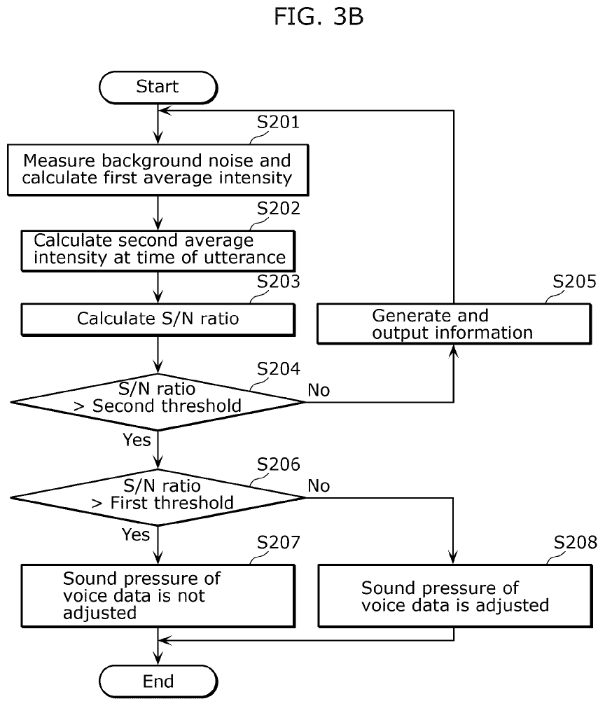

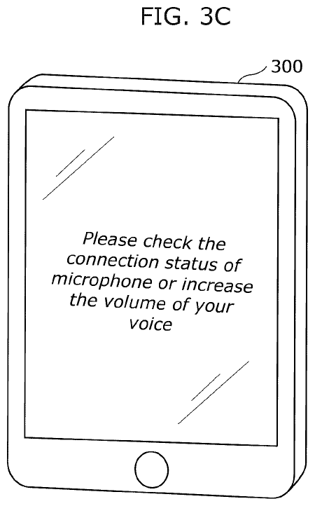

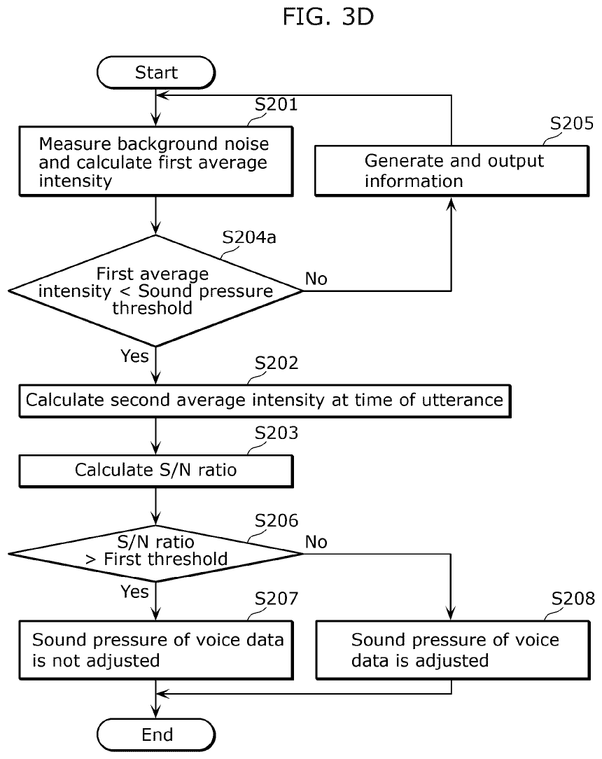

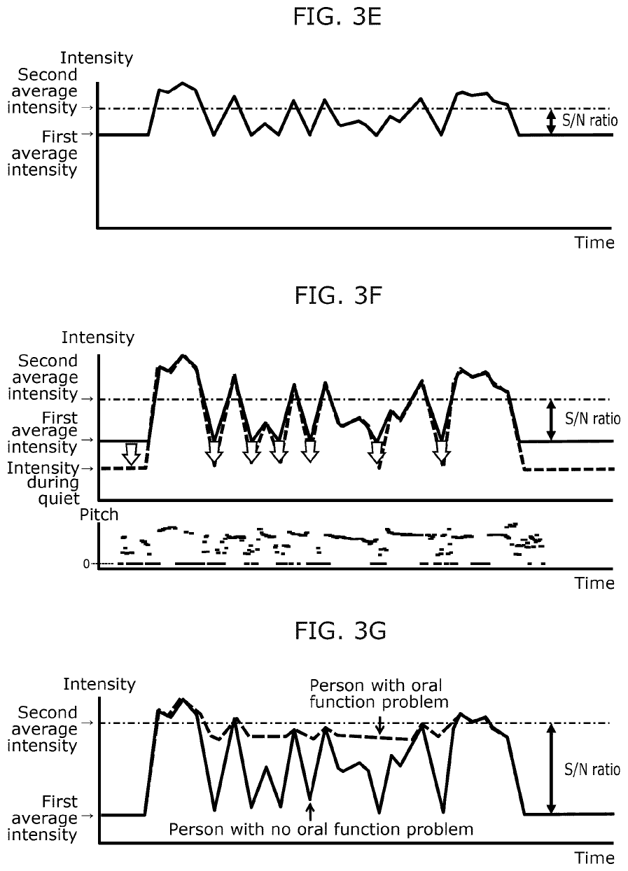

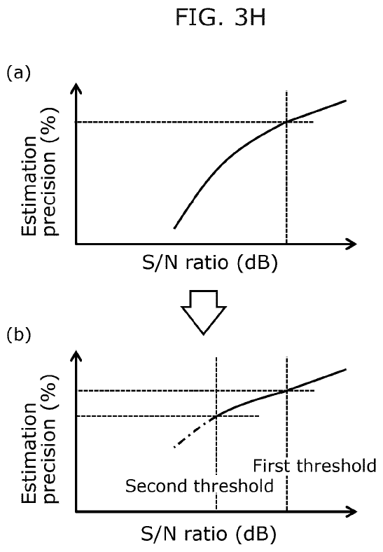

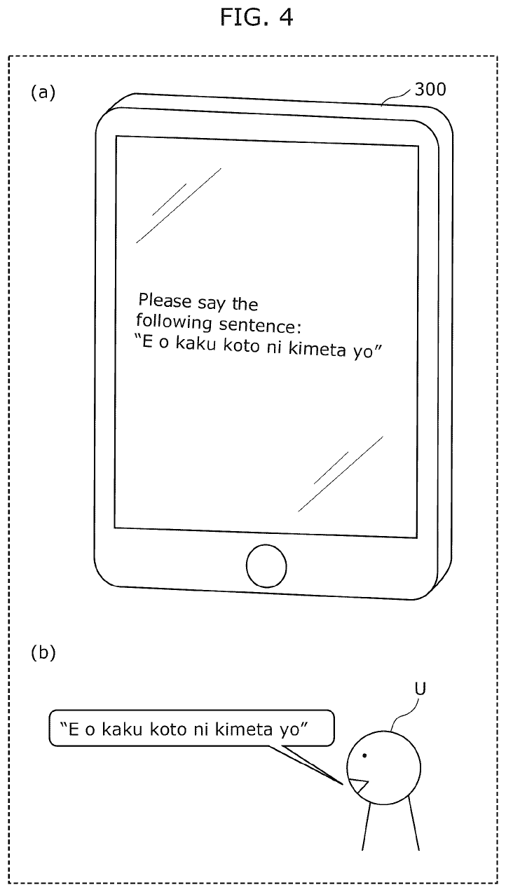

FIG. 3A is a flowchart illustrating a processing procedure for evaluating oral function of evaluatee U using the oral function evaluation method according to the embodiment. FIG. 4 is a diagram illustrating an outline of a method for obtaining a voice of evaluatee U using the oral function evaluation method.

First, the instructor instructs evaluatee U to utter a syllable or a fixed sentence that includes (i) two or more morae including a change in the first formant frequency or a change in the second formant frequency or (ii) at least one of a flap, a plosive, a voiceless sound, a double consonant, or a fricative (step S101). For example, in step S101, the instructor obtains image data on an instruction image stored in storage 170 and indicating an instruction to evaluatee U, and outputs the image data to mobile terminal 300. With this, as illustrated in (a) of FIG. 4, the instruction image indicating an instruction to evaluatee U is displayed on mobile terminal 300. It should be noted that although “E o kaku koto ni kimeta yo” is shown in (a) of FIG. 4 as an example of the fixed sentence, an instruction to utter a fixed sentence such as “Hana saka jiisan to saru kani kassen”, “Hanabi no e o kaku”, or “Himawari ga saita” may be provided. Alternatively, an instruction to utter syllables such as “ippai,” “ittai,” “ikkai,” “pattan,” “kappa,” “shippo,” “kikkari,” or “katteni” may be provided. Alternatively, an instruction to utter syllables such as “kara,” “sara,” “chara,” “jara,” “shara,” “kyara,” or “pura” may be provided. Alternatively, an instruction to utter syllables such as “aei,” “iea,” “ai,” “ia,” “kakeki,” “kikeka,” “naneni,” “chiteta,” “papepi,” “pipepa,” “katepi,” “chipeka,” “kaki,” “tachi,” “papi,” “misa,” “rari,” “wani,” “niwa,” “eo,” “io,” “iu,” “teko,” “kiro,” “teru”, “peko,” “memo,” or “emo” may be provided. The instruction to utter syllables may be an instruction to repeatedly utter such syllables as described above.

The instructor may obtain voice data on an instruction voice that is stored in storage 170 and indicates an instruction to evaluatee U, and output the voice data to mobile terminal 300 so as to provide the above-described instruction using the instruction voice that instructs evaluatee U to utter a syllable or a fixed sentence, without using the instruction image that instructs evaluatee U to utter a syllable or a fixed sentence. Alternatively, an evaluating person (a family member, a doctor, etc.) who wishes to evaluate oral function of evaluatee U may provide the above-described instruction to evaluatee U using the voice of the evaluating person, without using the instruction image or the instruction voice that instructs evaluatee U to utter a syllable or a fixed sentence.

For example, the syllable or the fixed sentence uttered may include a combination of two or more vowels or a vowel and a consonant. Here, the combination of two or more vowels or a vowel and a consonant involves mouth opening and closing or back and forth tongue movement for utterance. “E o kaku koto ni kimeta yo” in Japanese is an example of such syllables or a fixed sentence. Uttering “e o” in “e o kaku koto ni kimeta yo” involves back and forth tongue movement, and uttering “kimeta” in “e o kaku koto ni kimeta yo” involves mouth opening and closing. The part “e o” in “e o kaku koto ni kimeta yo” includes second formant frequencies of the vowel “e” and the vowel “o,” and includes an amount of change in the second formant frequency because the vowel “e” and the vowel “o” adjoin each other. This part also includes a change over time in the second formant frequency. The part “kimeta” in “e o kaku koto ni kimeta yo” includes first formant frequencies of the vowel “i,” the vowel “e,” and the vowel “a,” and includes amounts of change in the first formant frequency because the vowel “i,” the vowel “e,” and the vowel “a” adjoin one another. This part also includes changes over time in the first formant frequency. Uttering “e o kaku koto ni kimeta yo” enables extraction of prosody features such as sound pressure differences, the first formant frequencies, the second formant frequencies, the amounts of change in the first formant frequency, the amounts of change in the second formant frequency, the changes over time in the first formant frequency, the changes over time in the second formant frequency, the speech rate, and the like.

For example, the fixed sentence uttered may include repetition of syllables including a flap and a consonant different from the flap. “Karakarakara . . . ” in Japanese is an example of such a fixed sentence. Repeatedly uttering “karakarakara . . . ” enables extraction of prosody features such as sound pressure differences, changes over time in sound pressure difference, changes over time in sound pressure, the number of repetitions, and the like.

For example, the syllable or the fixed sentence uttered may include at least one combination of a vowel and a plosive. “Ittai” in Japanese is an example of such syllables. Uttering “ittai” enables extraction of prosody features such as sound pressure differences, a time length of a plosive (a time length between vowels), and the like.

Incidentally, the prosody feature of the sound pressure difference is easily affected by background noise, and thus, the prosody feature of the sound pressure difference may adversely affect the accuracy of the calculation of an estimate value especially in a sound collection environment with a relatively low S/N ratio. In view of the above, according to the present invention, the sound pressure of voice data is adjusted according to the S/N ratio calculated by S/N ratio calculator 115 so that the feature of the sound pressure difference calculated (extracted) becomes appropriate. According to the present invention, by making such an adjustment, an appropriate prosody feature of the sound pressure difference is calculated, thereby making it possible to calculate an estimate value with reduced possibility of an inappropriate prosodic feature of the sound pressure difference adversely affecting the accuracy of the calculation of an estimate value.

Operation such as specific processing performed for this purpose will now be described with reference to FIG. 3B through FIG. 3H. FIG. 3B is a flowchart illustrating a processing procedure related to voice data to be used in the oral function evaluation method according to the embodiment. FIG. 3C is a diagram illustrating an example of information output in the oral function evaluation method according to the embodiment. FIG. 3D is a flowchart illustrating a processing procedure related to voice data to be used in the oral function evaluation method according to another example of the embodiment. FIG. 3E through FIG. 3G are graphs for describing adjustment of the sound pressure of voice data according to the embodiment. FIG. 3H shows graphs each illustrating a relationship between adjustment of sound pressure in the oral function evaluation method according to the embodiment and accuracy (estimation precision).

As illustrated in FIG. 3B, in order to calculate the S/N ratio, S/N ratio calculator 115 measures background noise and calculates the first average intensity (sound pressure) of the background noise only (step S201). For the measurement of the background noise, it suffices so long as a sound collected in a period in which evaluatee U does not utter a voice is extracted and used. For example, as described above, when evaluatee U is uttering an instructed syllable or fixed sentence, a sound may be extracted in a background noise period before or after evaluatee U utters the syllable or the fixed sentence, or if the fixed sentence includes a pause, the pause may be regarded as the background noise period and a sound may be extracted during the pause.

Subsequently, in order to calculate the S/N ratio, S/N ratio calculator 115 calculates the second average intensity (sound pressure) at the time of the utterance of evaluatee U (step S202). Here, a sound included in the utterance of the instructed syllable or fixed sentence may be used, or evaluatee U may be instructed to separately utter an arbitrary syllable or fixed sentence for sound collection. Alternatively, if evaluatee U is in a situation of having a conversation with someone immediately before the evaluation of oral function, the first average intensity and the second average intensity may be calculated utilizing that situation.

S/N ratio calculator 115 subsequently calculates the S/N ratio by calculating the ratio of the second average intensity to the first average intensity (step S203). Here, the calculated S/N ratio is output to information outputter 180. Information outputter 180 then determines whether the S/N ratio is greater than a second threshold (step S204). When the S/N ratio is determined to be less than or equal to the second threshold (No in S204), information outputter 180 generates and outputs information for improving the sound collection environment so as to increase the S/N ratio (step S205).

For example, FIG. 3C illustrates, as an example of the case where such information is output, mobile terminal 300 displaying “Please check the connection status of microphone or increase the volume of your voice.” By outputting the information in such a manner, an instruction is provided to increase the S/N ratio by at least one of: reducing the background noise, i.e., decreasing the first average intensity; or increasing the volume of the evaluatee's voice, i.e., increasing the second average intensity. It should be noted that mobile terminal 300 may display “Please change the location for sound collection” so as to reduce the environmental sound when the evaluatee speaks.

As another example that achieves the same advantageous effect, the processing according to the flowchart illustrated in FIG. 3D may be performed. FIG. 3D is the same as FIG. 3B except that in FIG. 3D, instead of step S204, step S204a is performed after step S201 and before step S202. In this step, whether or not to output information is determined simply based on the loudness of the background noise, without calculating the S/N ratio. Specifically, in step S204a, whether or not the first average intensity is less than a sound pressure threshold is determined. When the first average intensity is less than the sound pressure threshold (Yes in S204a), steps S202 and S203 are performed, and the processing proceeds to step S206. On the other hand, when the first average intensity is greater than or equal to the sound pressure threshold (No in S204a), the processing proceeds to step S205 in which information outputter 180 generates and outputs information for improving the sound collection environment so as to increase the S/N ratio. This example is more advantageous than the example illustrated in FIG. 3B in that whether or not to output information can be determined simply based on the first average intensity only, without calculating the S/N ratio. That is to say, it is advantageous in that the appropriateness of the sound collection environment in terms of the amount of noise can be determined before instructing evaluatee U to speak for the calculation of the second average intensity.

Returning to FIG. 3B, when the S/N ratio is determined to be greater than the second threshold (Yes in S204) (or after step S203 in FIG. 3D), information outputter 180 does nothing in particular and proceeds to step S206. Specifically, the calculated S/N ratio is also output to sound pressure adjuster 116. Sound pressure adjuster 116 determines whether the S/N ratio is greater than a first threshold (step S206). When the S/N ratio is determined to be less than or equal to the first threshold (No in S206), sound pressure adjuster 116 adjusts the sound pressure of the voice data (step S208) and ends the processing. On the other hand, when the S/N ratio is determined to be greater than the first threshold (Yes in S206), sound pressure adjuster 116 does not adjust the sound pressure of the voice data (step S207) and ends the processing.

In such a manner, the sound pressure of the voice data is adjusted (or not adjusted) according to the S/N ratio, and the resulting voice data is provided for extraction of a prosody feature.

The adjustment of the sound pressure will now be described with reference to FIG. 3E to FIG. 3F. FIG. 3E illustrates a transition of change in the intensity of voice data with respect to time when the S/N ratio is less than the second threshold (or when the first average intensity is greater than the sound pressure threshold). In the example illustrated in FIG. 3E, the value of the S/N ratio is so small that an appropriate prosody feature cannot be extracted even if the sound pressure is adjusted, and therefore, information is output from information outputter 180 in order to improve the sound collection environment. Thus, in the case of the voice data illustrated in FIG. 3E, neither the extraction of a prosody feature nor the evaluation of oral function is performed.

FIG. 3F illustrates a transition of change in the intensity of voice data with respect to time (upper row) and a transition of change in the fundamental frequency (pitch) with respect to time (lower row) when the S/N ratio is greater than or equal to the second threshold and less than the first threshold. In the example illustrated in FIG. 3F, sound-pressure-adjusted voice data (dashed line) is generated by adjusting the sound pressure of the voice data obtained (solid line). As illustrated in the figure, the sound pressure adjustment is performed at the timing when the sound pressure indicates a local minimum value and the fundamental frequency indicates zero (white arrows in the figure) in the voice data. This makes it possible to appropriately adjust the sound pressure at the timing when there is no sound and the intensity of the sound is at its local minimum.

In the adjustment of the sound pressure, a sound pressure equivalent to the difference between the intensity of a sound during quiet and the first average intensity is subtracted from the sound pressure at a local minimum value at the above timing. As a result, only the local minimum value is decreased and the influence of the background noise is thereby reduced without causing a significant change around a local maximum point in the voice data. Accordingly, a sound pressure difference etc. characterized by the difference between a local maximum value and a local minimum value can be extracted as a more appropriate feature. It should be noted that as the intensity of a sound during quiet described above, a virtual intensity set in advance may be stored in storage 170 or the like and read out and used at the time of the sound pressure adjustment. Also, the lowest value of the intensity actually measured in the past under the same sound collection condition may be used as the intensity of a sound during quiet.

In contrast, FIG. 3G illustrates a transition of change in the intensity of voice data with respect to time when the S/N ratio is greater than or equal to the first threshold. In the example illustrated in FIG. 3G, an example of voice data of a person with no oral function problem is shown with a solid line, and an example of voice data of a person with an oral function problem is shown with a dashed line. When the S/N ratio is greater than or equal to the first threshold, adjusting the sound pressure in an unthoughtful manner may cause the voice data of a person with an oral function problem to be misinterpreted as voice data of a person with no oral function problem. Therefore, it is effective to make a setting that prohibits the sound pressure adjustment when the S/N ratio is greater than or equal to the first threshold.

In view of the above, the second threshold may be empirically or experimentally determined so that the second threshold is set to a value greater than the S/N ratio at which no appropriate prosody feature can be extracted even if the sound pressure is adjusted. The first threshold may also be determined empirically or experimentally to prevent the sound pressure adjustment on voice data of a person with an oral function problem. For example, in FIG. 3H, graph (a) illustrates the relationship between the S/N ratio and estimation precision when a prosody feature is extracted from voice data as-is without taking the S/N ratio into consideration, and graph (b) illustrates the relationship between the S/N ratio and estimation precision when a prosody feature is extracted from voice data resulting from the sound pressure adjustment performed according to the S/N ratio.

As illustrated in FIG. 3H, when the S/N ratio is greater than or equal to the first threshold, the estimation precision is the same in both graphs (a) and (b). However, in the range in which the S/N ratio is less than the first threshold and greater than or equal to the second threshold, the estimation precision is lower in graph (a) of FIG. 3H than in graph (b) of FIG. 3H because in graph (a) of FIG. 3H, a prosody feature related to a sound pressure affected by the background noise reduces the estimation precision. When the S/N ratio is less than the second threshold, an instruction to increase the S/N ratio is provided. As a result, before the calculation of an estimate value takes place, the environment for the sound collection is changed to an environment with an improved S/N ratio, and processing is performed again from the obtaining of voice data. Accordingly, as shown by the dash-dot-dash line in graph (b) of FIG. 3H, an estimate value is less likely to be calculated with low estimation precision. However, in some cases, the estimation precision is higher than that in graph (a) of FIG. 3H even in a state in which the S/N ratio is less than the second threshold, so it may be still useful to calculate an estimate value in such a state.

Returning to the description of FIG. 3A, the voice data may be obtained by collecting a voice of evaluatee U uttering a syllable or a fixed sentence at least twice at different speech rates. For example, evaluatee U is instructed to utter “e o kaku koto ni kimeta yo” at his/her usual speed and at a faster speed. The maintenance level of the state of oral function can be estimated by evaluatee U uttering “e o kaku koto ni kimeta yo” at his/her usual speed and at a faster speed.

Next, as illustrated in FIG. 3A, obtainer 110 obtains, via mobile terminal 300, the voice data of evaluatee U instructed in step S101 (step S102). As illustrated in (b) of FIG. 4, in step S102, for example, evaluatee U utters syllables or a fixed sentence such as “e o kaku koto ni kimeta yo” toward mobile terminal 300. Obtainer 110 obtains, as the voice data, the syllables or the fixed sentence uttered by evaluatee U.

Next, when the S/N ratio is greater than the first threshold, extractor 120 extracts a prosody feature from the voice data obtained by obtainer 110, whereas when the S/N ratio is less than or equal to the first threshold, extractor 120 extracts a prosody feature from the voice data output by sound pressure adjuster 116 (step S103).

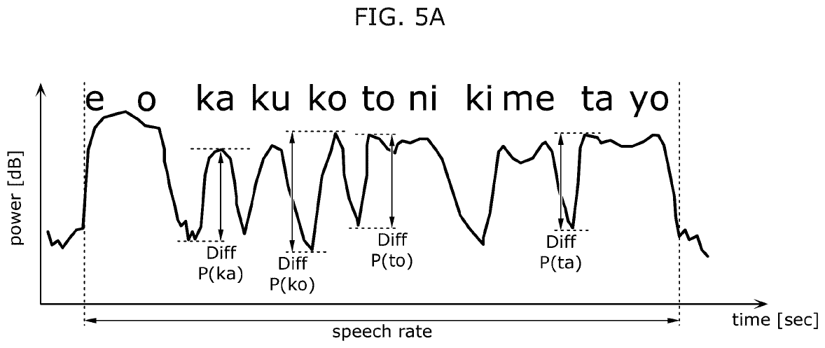

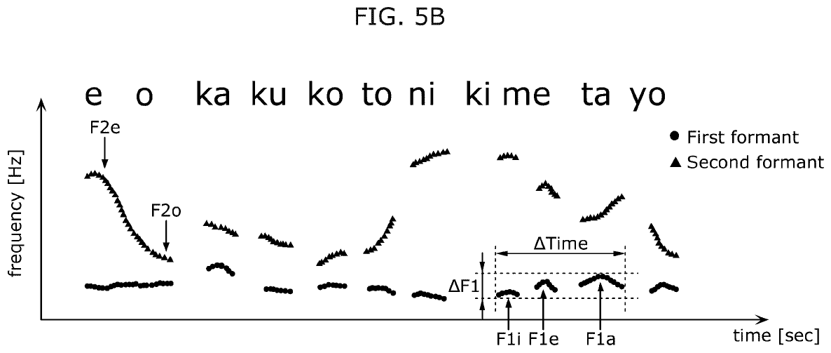

For example, when the voice data obtained by obtainer 110 is voice data obtained from a voice of evaluatee U uttering “e o kaku koto ni kimeta yo,” extractor 120 extracts, as the prosody features, sound pressure differences, the first formant frequencies, the second formant frequencies, the amounts of change in the first formant frequency, the amounts of change in the second formant frequency, the changes over time in the first formant frequency, the changes over time in the second formant frequency, and the speech rate. This will be described with reference to FIG. 5A and FIG. 5B.

FIG. 5A is a graph illustrating an example of voice data indicating a voice of evaluatee U uttering “e o kaku koto ni kimeta yo.” In the graph illustrated in FIG. 5A, the horizontal axis indicates time, and the vertical axis indicates power (sound pressure). It should be noted that the power indicated on the vertical axis of the graph in FIG. 5A is expressed in decibels (dB).

In the graph illustrated in FIG. 5A, changes in sound pressure corresponding to “e,” “o,” “ka,” “ku,” “ko,” “to,” “ni,” “ki,” “me,” “ta,” “yo” are recognized. In step S102 shown in FIG. 3A, obtainer 110 obtains from evaluatee U the voice data illustrated in FIG. 5A. The S/N ratio calculator calculates the S/N ratio using the voice data obtained, and according to the S/N ratio calculated, either the voice data obtained by obtainer 110 or the voice data output by sound pressure adjuster 116 is determined as the voice data to be provided to extractor 120.

For example, extractor 120 extracts, in step S103 shown in FIG. 3A, sound pressures of “k” and “a” in “ka,” sound pressures of “k” and “o” in “ko,” sound pressures of “t” and “o” in “to,” and sound pressures of “t” and “a” in “ta” included in the voice data illustrated in FIG. 5A, with a known method. From the sound pressures of “k” and “a” extracted, extractor 120 extracts sound pressure difference Diff_P(ka) between “k” and “a” as a prosody feature. Likewise, extractor 120 extracts sound pressure difference Diff_P(ko) between “k” and “o,” sound pressure difference Diff_P(to) between “t” and “o,” and sound pressure difference Diff_P(ta) between “t” and “a” as prosody features. For example, based on a sound pressure difference, oral function regarding swallowing force (pressure of the tongue in contact with the palate) or bolus formation ability can be evaluated. In addition, based on a sound pressure difference including “k,” oral function regarding an ability to prevent food and drink from flowing into the throat can be evaluated.

FIG. 5B is a graph illustrating an example of changes in formant frequencies of a voice of evaluatee U uttering “e o kaku koto ni kimeta yo.” Specifically, FIG. 5B is a graph for describing an example of changes in the first formant frequency and changes in the second formant frequency.

The first formant frequency is a peak frequency of the amplitude of a human voice that appears first from the low-frequency side. The first formant frequency is known for its tendency to reflect a feature regarding mouth opening and closing. The second formant frequency is a peak frequency of the amplitude of a human voice that appears second from the low-frequency side. The second formant frequency is known for its tendency to reflect an influence regarding back and forth tongue movement.

From the voice data indicating the voice uttered by evaluatee U, extractor 120 extracts a first formant frequency and a second formant frequency of each of the vowels, as prosody features. For example, extractor 120 extracts second formant frequency F2e corresponding to the vowel “e” and second formant frequency F2o corresponding to the vowel “o” in “e o,” as the prosody features. In addition, for example, extractor 120 extracts first formant frequency F1i corresponding to the vowel “i,” first formant frequency F1e corresponding to the vowel “e,” and first formant frequency F1a corresponding to the vowel “a” in “kimeta,” as the prosody features.

Extractor 120 further extracts amounts of change in the first formant frequency and amounts of change in the second formant frequency of a string of consecutive vowels, as the prosody features. For example, extractor 120 extracts an amount of change between second formant frequency F2e and second formant frequency F2o (F2e−F2o) and amounts of change between first formant frequency F1i, first formant frequency F1e, and first formant frequency F1a (F1e−F1i, F1a−F1e, and F1a−F1i), as the prosody features.

Extractor 120 further extracts changes over time in the first formant frequency and changes over time in the second formant frequency of a string of consecutive vowels, as prosody features. For example, extractor 120 extracts a change over time from second formant frequency F2e to second formant frequency F2o and a change over time from first formant frequency F1i through first formant frequency F1e to first formant frequency F1a, as the prosody features. FIG. 5B illustrates an example of the change over time from first formant frequency F1i through first formant frequency F1e to first formant frequency F1a, and the change over time is ΔF1/ΔTime. Here, ΔF1 is F1a−F1i.

For example, based on the second formant frequency, an amount of change in the second formant frequency, or a change over time in the second formant frequency, oral function regarding movement of gathering food (tongue movement in all directions) can be evaluated. In addition, for example, based on the first formant frequency, an amount of change in the first formant frequency, or a change over time in the first formant frequency, oral function regarding an ability to chew food can be evaluated. In addition, based on a change over time in the first formant frequency, oral function regarding an ability to move the mouth quickly can be evaluated.

Extractor 120 may extract the speech rate as a prosody feature as illustrated in FIG. 5A. For example, extractor 120 may extract, as a prosody feature, a time length from the start to the end of the utterance of “e o kaku koto ni kimeta yo” by evaluatee U. Also, for example, extractor 120 may extract, as a prosody feature, a time length from the start to the end of utterance of a given part of “e o kaku koto ni kimeta yo” rather than the time length taken to finish the utterance of the entire “e o kaku koto ni kimeta yo.” Furthermore, for example, extractor 120 may extract, as a prosody feature, an average time length taken to utter the entire “e o kaku koto ni kimeta yo” or one or more words in a given part of “e o kaku koto ni kimeta yo.” For example, based on the speech rate, oral function regarding movement of swallowing, movement of gathering food, or tongue dexterity can be evaluated.

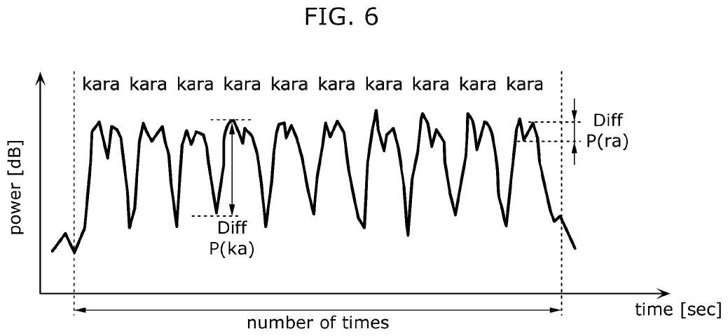

For example, when the voice data obtained by obtainer 110 is voice data obtained from a voice of evaluatee U repeatedly uttering “karakarakara . . . ,” extractor 120 extracts changes over time in sound pressure difference as a prosody feature. This will be described with reference to FIG. 6.

FIG. 6 is a graph illustrating an example of voice data indicating a voice of evaluatee U repeatedly uttering “karakarakara . . . .” In the graph illustrated in FIG. 6, the horizontal axis indicates time, and the vertical axis indicates power (sound pressure). It should be noted that the power indicated on the vertical axis of the graph in FIG. 6 is expressed in decibels (dB).

In the graph illustrated in FIG. 6, changes in sound pressure corresponding to “ka” and “ra” are recognized. In step S102 shown in FIG. 3A, obtainer 110 obtains from evaluatee U the voice data illustrated in FIG. 6. For example, extractor 120 extracts, in step S103 shown in FIG. 3A, sound pressures of “k” and “a” in “ka” and sound pressures of “r” and “a” in “ra” included in the voice data illustrated in FIG. 6, with a known method. From the sound pressures of “k” and “a” extracted, extractor 120 extracts sound pressure difference Diff_P(ka) between “k” and “a” as a prosody feature. Likewise, extractor 120 extracts sound pressure difference Diff_P(ra) between “r” and “a” as a prosody feature. For example, extractor 120 extracts sound pressure difference Diff_P(ka) and sound pressure difference Diff_P(ra) as prosody features from each of repeatedly uttered “kara.” Extractor 120 subsequently extracts a change over time in sound pressure difference Diff_P(ka) as a prosody feature from each of sound pressure differences Diff_P(ka) extracted and extracts a change over time in sound pressure difference Diff_P(ra) as a prosody feature from each of sound pressure differences Diff_P(ra) extracted. For example, based on the changes over time in the sound pressure difference, oral function regarding movement of swallowing, movement of gathering food, or an ability to chew food can be evaluated.

It should be noted that extractor 120 may extract a change over time in sound pressure as a prosody feature. For example, in each of “kara” repeated in the utterance of “karakarakara . . . ,” a change over time in minimum sound pressure (sound pressure of “k”) may be extracted, a change over time in maximum sound pressure (sound pressure of “a”) may be extracted, or a change over time in sound pressure between “ka” and “ra” (sound pressure of “r”) may be extracted. For example, based on the changes over time in sound pressure, oral function regarding movement of swallowing, movement of gathering food, or an ability to chew food can be evaluated.

As illustrated in FIG. 6, extractor 120 may also extract, as a feature, the number of repetitions that is the number of times evaluatee U was able to utter “kara” per given time period. The given time period is not limited to a particular time period. For example, the given time period is five seconds. For example, based on the number of repetitions per given time period, oral function regarding movement of swallowing or movement of gathering food can be evaluated.

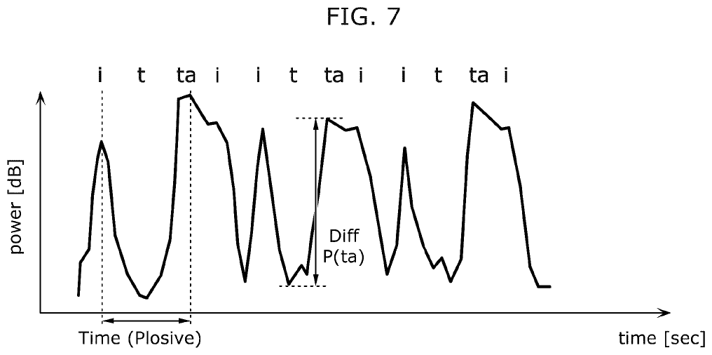

For example, when the voice data obtained by obtainer 110 is voice data obtained from a voice of evaluatee U uttering “ittai,” extractor 120 extracts a sound pressure difference and a time length of a plosive as prosody features. This will be described with reference to FIG. 7.

FIG. 7 is a graph illustrating an example of voice data indicating a voice of evaluatee U uttering “ittai.” Here, an example of voice data indicating a voice of evaluatee U repeatedly uttering “ittaiittai . . . ” is illustrated. In the graph illustrated in FIG. 7, the horizontal axis indicates time, and the vertical axis indicates power (sound pressure). It should be noted that the power indicated on the vertical axis of the graph in FIG. 7 is expressed in decibels (dB).

In the graph illustrated in FIG. 7, changes in sound pressure corresponding to “i,” “t,” “ta,” and “i” are recognized. In step S102 shown in FIG. 3A, obtainer 110 obtains from evaluatee U the voice data illustrated in FIG. 7. For example, extractor 120 extracts, in step S103 shown in FIG. 3A, sound pressures of “t” and “a” in “ta” included in the voice data illustrated in FIG. 7, with a known method. From the sound pressures of “t” and “a” extracted, extractor 120 extracts sound pressure difference Diff_P(ta) between “t” and “a” as a prosody feature. For example, based on the sound pressure difference, oral function regarding swallowing force or bolus formation ability can be evaluated. Extractor 120 also extracts a time length of a plosive Time(i-ta) (a time length of a plosive between “i” and “ta”) as a prosody feature. For example, based on the time length of a plosive, oral function regarding movement of swallowing, movement of gathering food, or stable tongue movement can be evaluated.

It should be noted that although Japanese syllables and fixed sentences have been given as examples of the syllable or fixed sentence to be uttered, the syllable or fixed sentence to be uttered is not limited to Japanese and may be in any language.

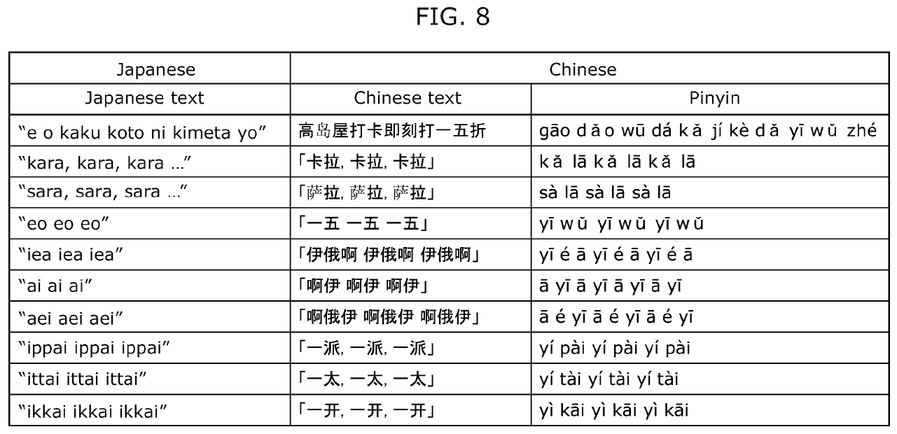

FIG. 8 is a table showing an example of syllables and fixed sentences in Japanese and syllables and fixed sentences in Chinese that are similar in tongue movement or degree of mouth opening and closing when pronounced.

There are various languages in the world, and there are pronunciations that are similar in tongue movement or degree of mouth opening and closing across different languages. For example, a Chinese sentence [symbol] [symbol] [symbol]
(hereinafter written as gao dao wu da ka ji ke da yi wu zhe) is similar to a Japanese sentence “e o kaku koto ni kimeta yo” in tongue movement or degree of mouth opening and closing when pronounced and thus enables extraction of prosody features similar to prosody features of the Japanese sentence “e o kaku koto ni kimeta yo.” It should be noted that tonal markers are omitted in the present specification. FIG. 8 shows, for reference, some examples of pairs of syllables or fixed sentences in Japanese and Chinese that are similar in tongue movement or degree of mouth opening and closing when pronounced.

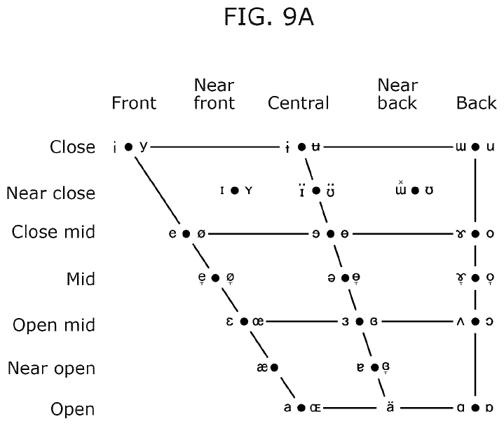

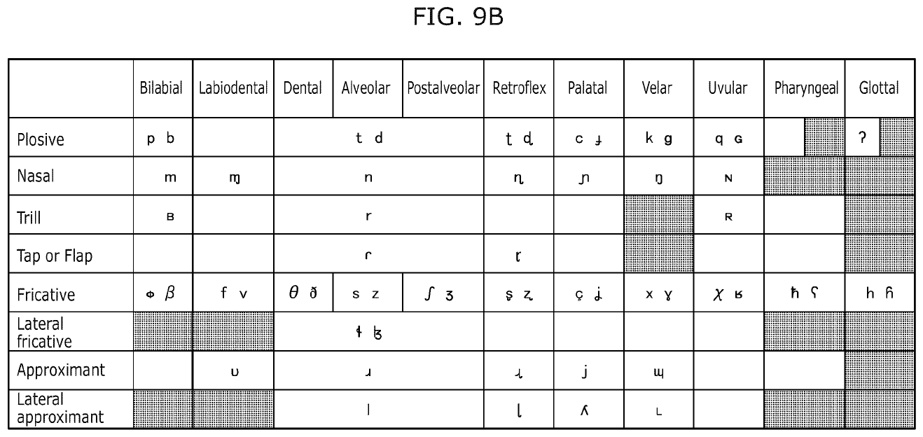

With reference to FIG. 9A and FIG. 9B, the following briefly describes the fact that there are pronunciations similar in tongue movement or degree of mouth opening and closing across different languages among various languages spoken in the world.

FIG. 9A is a diagram illustrating international phonetic alphabet symbols of vowels.

FIG. 9B is a table illustrating international phonetic alphabet symbols of consonants.

In a position relationship of the international phonetic alphabet symbols of vowels illustrated in FIG. 9A, the horizontal direction indicates back and forth tongue movement where symbols close to each other are similar in back and forth tongue movement, and the vertical direction indicates a degree of mouth opening and closing where symbols close to each other are similar in degree of mouth opening and closing. In the table of international phonetic alphabet symbols of consonants illustrated in FIG. 9B, the horizontal direction indicates parts from the lips to the throat used in pronunciation, and the same sound can be pronounced using the same part based on international phonetic alphabet symbols present in the same cell of the table. For this reason, the present invention is applicable to various languages spoken in the world.

For example, when a large mouth opening and closing is intended, syllables or a fixed sentence is set to include consecutive international phonetic alphabet symbols that are away from each other in the vertical direction illustrated in FIG. 9A (e.g., “i” and “a”). Accordingly, an amount of change in the first formant frequency can be increased as a prosody feature. For example, when large back and forth tongue movement is intended, syllables or a fixed sentence is set to include consecutive international phonetic alphabet symbols that are away from each other in the horizontal direction illustrated in FIG. 9A (e.g., “i” and “u”). Accordingly, an amount of change in the second formant frequency can be increased as a prosody feature.

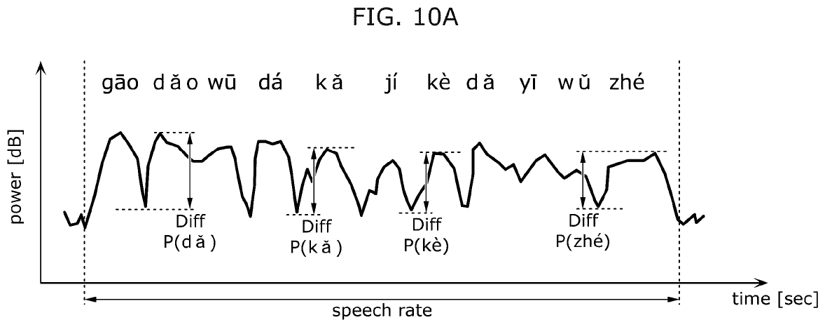

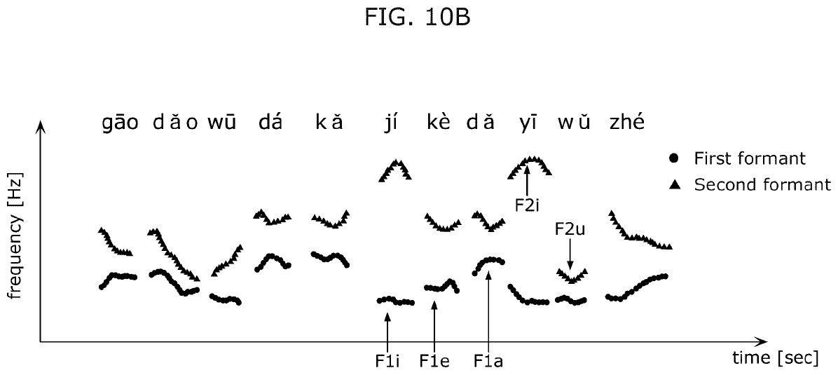

For example, when the voice data obtained by obtainer 110 is voice data obtained from a voice of evaluatee U uttering “gao dao wu da ka ji ke da yi wu zhe,” extractor 120 extracts, as prosody features, sound pressure differences, the first formant frequencies, the second formant frequencies, the amounts of change in the first formant frequency, the amounts of change in the second formant frequency, the changes over time in the first formant frequency, the changes over time in the second formant frequency, and the speech rate. This will be described with reference to FIG. 10A and FIG. 10B.

FIG. 10A is a graph illustrating an example of voice data indicating a voice of evaluatee U uttering “gao dao wu da ka ji ke da yi wu zhe.” In the graph illustrated in FIG. 10A, the horizontal axis indicates time, and the vertical axis indicates power (sound pressure). It should be noted that the power indicated on the vertical axis of the graph in FIG. 10A is expressed in decibels (dB).

In the graph illustrated in FIG. 10A, changes in sound pressure corresponding to “gao,” “dao,” “wu,” “da,” “ka,” “ji,” “ke,” “da,” “yi,” “wu,” and “zhe” are recognized. In step S102 shown in FIG. 3A, obtainer 110 obtains from evaluatee U the voice data illustrated in FIG. 10A. For example, extractor 120 extracts, in step S103 shown in FIG. 3A, sound pressures of “d” and “a” in “dao,” sound pressures of “k” and “a” in “ka,” sound pressures of “k” and “e” in “ke,” and sound pressures of “zh” and “e” in “zhe” included in the voice data illustrated in FIG. 10A, with a known method. From the sound pressures of “d” and “a” extracted, extractor 120 extracts sound pressure difference Diff_P(da) between “d” and “a” as a prosody feature. Likewise, extractor 120 extracts, as prosody features, sound pressure difference Diff_P(ka) between “k” and “a,” sound pressure difference Diff_P(ke) between “k” and “e,” and sound pressure difference Diff_P(zhe) between “zh” and “e.” For example, based on the sound pressure difference, oral function regarding swallowing force or bolus formation ability can be evaluated. In addition, based on the sound pressure difference including “k,” oral function regarding an ability to prevent food and drink from flowing into the throat can be evaluated.

FIG. 10B is a graph illustrating an example of changes in formant frequencies of a voice of evaluatee U uttering “gao dao wu da ka ji ke da yi wu zhe.” Specifically, FIG. 10B is a graph for describing an example of changes in the first formant frequency and changes in the second formant frequency.

From the voice data indicating the voice uttered by evaluatee U, extractor 120 extracts the first formant frequency and the second formant frequency of each vowel, as prosody features. For example, extractor 120 extracts first formant frequency F1i corresponding to the vowel “i” in “ji,” first formant frequency F1e corresponding to the vowel “e” in “ke,” and first formant frequency F1a corresponding to the vowel “a” in “da,” as prosody features. In addition, for example, extractor 120 extracts second formant frequency F2i corresponding to the vowel “i” in “yi,” and second formant frequency F2u corresponding to the vowel “u” in “wu,” as prosody features.

Extractor 120 further extracts amounts of change in the first formant frequency and amounts of change in the second formant frequency of a string of consecutive vowels, as prosody features. For example, extractor 120 extracts amounts of change between first formant frequency F1i, first formant frequency F1e, and first formant frequency F1a (F1e−F1i, F1a−F1e, and F1a−F1i) and an amount of change between second formant frequency F2i and second formant frequency F2u (F2i−F2u), as prosody features.

Extractor 120 further extracts changes over time in the first formant frequency and changes over time in the second formant frequency of a string of consecutive vowels, as prosody features. For example, extractor 120 extracts a change over time from first formant frequency F1i through first formant frequency F1e to first formant frequency F1a and a change over time from second formant frequency F2i to second formant frequency F2u, as prosody features.

For example, based on the second formant frequency, an amount of change in the second formant frequency, or a change over time in the second formant frequency, oral function regarding movement of gathering food can be evaluated. In addition, for example, based on the first formant frequency, an amount of change in the first formant frequency, or a change over time in the first formant frequency, oral function regarding an ability to chew food can be evaluated. In addition, based on a change over time in the first formant frequency, oral function regarding an ability to move the mouth quickly can be evaluated.

Extractor 120 may also extract the speech rate as a prosody feature as illustrated in FIG. 10A. For example, extractor 120 may extract, as a prosody feature, a time length from the start to the end of the utterance of “gao dao wu da ka ji ke da yi wu zhe” by evaluatee U. Also, for example, extractor 120 may extract, as a prosody feature, a time length from the start to the end of utterance of a given part of “gao dao wu da ka ji ke da yi wu zhe” rather than the time length taken to finish the utterance of the entire “gao dao wu da ka ji ke da yi wu zhe.” Furthermore, for example, extractor 120 may extract, as a prosody feature, an average time length taken to utter the entire “gao dao wu da ka ji ke da yi wu zhe” or one or more words in a given part of “gao dao wu da ka ji ke da yi wu zhe.” For example, based on the speech rate, oral function regarding movement of swallowing, movement of gathering food, or tongue dexterity can be evaluated.

Returning to the description of FIG. 3A, calculator 130 calculates an estimate value of oral function of evaluatee U, based on the prosody feature extracted and an oral function estimating equation calculated based on a plurality of training data items (step S104).

The oral function estimating equation is set in advance based on the results of evaluation performed on a plurality of subjects. Through a statistical analysis of voice features collected from utterances of the subjects and results of actual diagnoses on oral function of the subjects, the estimating equation is set in the form of a multiple regression equation or the like about correlations between the voice features and the results of the diagnoses. Depending on a voice feature selected to be used as a representative value, different types of estimating equations can be generated. The estimating equation can be generated in advance in this manner.

Alternatively, the estimating equation may be set using machine learning to express correlations between the voice features and the results of the diagnoses. Techniques of the machine learning include logistic regression, support vector machine (SVM), and random forest.

For example, an estimating equation can include a coefficient corresponding to an element of oral function and a variable that is substituted by a prosody feature extracted and is multiplied by the coefficient. Equations 1 through 5 shown below are examples of the estimating equation.

$$Estimate value of tongue fur adhesion = ( A 1 \times F 2 e ) + ( B 1 \times F 2 o ) + ( C 1 \times F 1 i ) + ( D 1 \times F 1 e ) + ( E 1 \times F 1 a ) + ( F 1 \times Diff\_P ( ka ) ) + ( G 1 \times Diff\_P ( ko ) ) + ( H 1 \times Diff\_P ( to ) ) + ( J 1 \times Diff\_P ( ta ) ) + ( K 1 \times Diff\_P ( ka ) ) + ( L 1 \times Diff\_P ( ra ) ) + ( M 1 \times Num ( kara ) ) + ( N 1 \times Diff\_P ( ta ) ) + ( P 1 \times Time ( i - ta ) ) + Q 1 \qquad ( Equation 1 )$$

$$Estimate value of oral mucous wetness = ( A 2 \times F 2 e ) + ( B 2 \times F 2 o ) + ( C 2 \times F 1 i ) + ( D 2 \times F 1 e ) + ( E 2 \times F 1 a ) + ( F 2 \times Diff\_P ( ka ) ) + ( G 2 \times Diff\_P ( ko ) ) + ( H 2 \times Diff\_P ( to ) ) + ( J 2 \times Diff\_P ( ta ) ) + ( K 2 \times Diff\_P ( ka ) ) + ( L 2 \times Diff\_P ( ra ) ) + ( M 2 \times Num ( kara ) ) + ( N 2 \times Diff\_P ( ta ) ) + ( P 2 \times Time ( i - ta ) ) + Q 2 \qquad ( Equation 2 )$$

$$Estimate value of occlusal force = ( A 3 \times F 2 e ) + ( B 3 \times F 2 o ) + ( C 3 \times F 1 i ) + ( D 3 \times F 1 e ) + ( E 3 \times F 1 a ) + ( F 3 \times Diff\_P ( ka ) ) + ( G 3 \times Diff\_P ( ko ) ) + ( H 3 \times Diff\_P ( to ) ) + ( J 3 \times Diff\_P ( ta ) ) + ( K 3 \times Diff\_P ( ka ) ) + ( L 3 \times Diff\_P ( ra ) ) + ( M 3 \times Num ( kara ) ) + ( N 3 \times Diff\_P ( ta ) ) + ( P 3 \times Time ( i - ta ) ) + Q 3 \qquad ( Equation 3 )$$

$$Estimate value of tongue pressure = ( A 4 \times F 2 e ) + ( B 4 \times F 2 o ) + ( C 4 \times F 1 i ) + ( D 4 \times F 1 e ) + ( E 4 \times F 1 a ) + ( F 4 \times Diff\_P ( ka ) ) + ( G 4 \times Diff\_P ( ko ) ) + ( H 4 \times Diff\_P ( to ) ) + ( J 4 \times Diff\_P ( ta ) ) + ( K 4 \times Diff\_P ( ka ) ) + ( L 4 \times Diff\_P ( ra ) ) + ( M 4 \times Num ( kara ) ) + ( N 4 \times Diff\_P ( ta ) ) + ( P 4 \times Time ( i - ta ) ) + Q 4 \qquad ( Equation 4 )$$

$$Estimate value of mastication function = ( A 5 \times F 2 e ) + ( B 5 \times F 2 o ) + ( C 5 \times F 1 i ) + ( D 5 \times F 1 e ) + ( E 5 \times F 1 a ) + ( F 5 \times Diff\_P ( ka ) ) + ( G 5 \times Diff\_P ( ko ) ) + ( H 5 \times Diff\_P ( to ) ) + ( J 5 \times Diff\_P ( ta ) ) + ( K 5 \times Diff\_P ( ka ) ) + ( L 5 \times Diff\_P ( ra ) ) + ( M 5 \times Num ( kara ) ) + ( N 5 \times Diff\_P ( ta ) ) + ( P 5 \times Time ( i - ta ) ) + Q 5 \qquad ( Equation 5 )$$

A1, B1, C1, . . . , P1, A2, B2, C2, . . . , P2, A3, B3, C3, . . . , P3, A4, B4, C4, . . . , P4, A5, B5, C5, . . . , P5 are coefficients, and are specifically coefficients corresponding to elements of oral function. For example, A1, B1, C1, . . . , P1 are coefficients corresponding to tongue fur adhesion which is one of the elements of oral function; A2, B2, C2, . . . , P2 are coefficients corresponding to oral mucous wetness which is one of the elements of oral function; A3, B3, C3, . . . , P3 are coefficients corresponding to occlusal force which is one of the elements of oral function; A4, B4, C4, . . . , P4 are coefficients corresponding to tongue pressure which is one of the elements of oral function; and A5, B5, C5, . . . , P5 are coefficients corresponding to mastication function which is one of the elements of oral function.

Q1 is a constant corresponding to tongue fur adhesion, Q2 is a constant corresponding to oral mucous wetness, Q3 is a constant corresponding to occlusal force, Q4 is a constant corresponding to tongue pressure, and Q5 is a constant corresponding to mastication function.

F2e multiplied by A1, A2, A3, A4, or A5 and F2o multiplied by B1, B2, B3, B4, or B5 are variables to be substituted by second formant frequencies that are prosody features extracted from utterance data on the utterance of “e o kaku koto ni kimeta yo” by evaluatee U. F1i multiplied by C1, C2, C3, C4, or C5, F1e multiplied by D1, D2, D3, D4, or D5, and F1a multiplied by E1, E2, E3, E4, or E5 are variables to be substituted by first formant frequencies that are prosody features extracted from utterance data on the utterance of “e o kaku koto ni kimeta yo” by evaluatee U. Diff_P(ka) multiplied by F1, F2, F3, F4, or F5, Diff_P(ko) multiplied by G1, G2, G3, G4, or G5, Diff_P(to) multiplied by H1, H2, H3, H4, or H5, and Diff_P(ta) multiplied by J1, J2, J3, J4, or J5 are variables to be substituted by sound pressure differences that are prosody features extracted from utterance data on the utterance of “e o kaku koto ni kimeta yo” by evaluatee U. Diff_P(ka) multiplied by K1, K2, K3, K4, or K5 and Diff_P(ra) multiplied by L1, L2, L3, L4, or L5 are variables to be substituted by sound pressure differences that are prosody features extracted from utterance data on the utterance of “kara” by evaluatee U. Num(kara) multiplied by M1, M2, M3, M4, or M5 is a variable to be substituted by the number of repetitions that is a prosody feature extracted from utterance data on the repeated utterance of “kara” by evaluatee U within a certain period. Diff_P(ta) multiplied by N1, N2, N3, N4, or N5 is a variable to be substituted by a sound pressure difference that is a prosody feature extracted from utterance data on the utterance of “ittai” by evaluatee U. Time(i-ta) multiplied by P1, P2, P3, P4, or P5 is a variable to be substituted by a time length of a plosive that is a prosody feature extracted from utterance data on the utterance of “ittai” by evaluatee U.

As shown in Equations 1 through 5 above, calculator 130, for example, calculates an estimate value for each of elements (e.g., tongue fur adhesion, oral mucous wetness, occlusal force, tongue pressure, and mastication function) of oral function of evaluatee U. It should be noted that these elements of oral function are mere examples, and it suffices so long as the elements of oral function include at least one of tongue fur adhesion, oral mucous wetness, occlusal force, tongue pressure, cheek pressure, the remaining number of teeth, swallowing function, or mastication function of evaluatee U.

In addition, for example, extractor 120 extracts a plurality of prosody features from voice data obtained by collecting a voice of evaluatee U uttering two or more types of syllables or two or more types of fixed sentences (e.g., “e o kaku koto ni kimeta yo,” “kara,” and “ittai” in Equations 1 through 5 shown above), and calculator 130 calculates an estimate value of oral function based on the plurality of prosody features extracted and one of the estimating equations. By substituting the plurality of prosody features extracted from the voice data on the two or more types of syllables or two or more types of fixed sentences into one of the estimating equations, calculator 130 can calculate the estimate value of oral function with high precision.

It should be noted that, although the linear expressions are shown as the estimating equations, the estimating equations may be multidimensional equations such as two-dimensional equations.

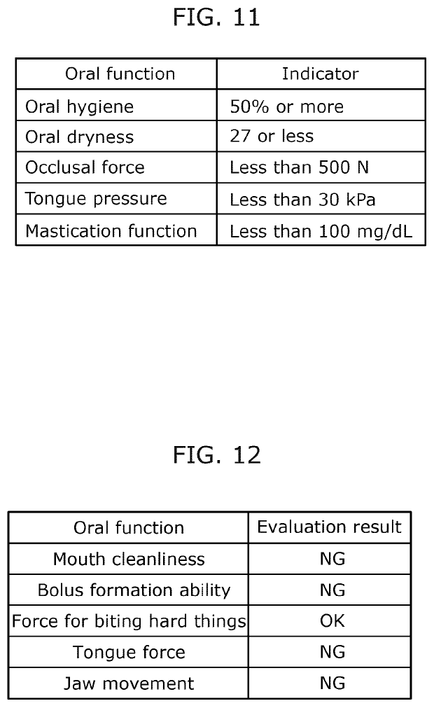

Next, evaluator 140 evaluates a deterioration state of oral function of evaluatee U by assessing, using an oral function evaluation indicator, the estimate value calculated by calculator 130 (step S105). For example, evaluator 140 evaluates a deterioration state of oral function of evaluatee U for each of the elements of oral function by assessing, using an oral function evaluation indicator determined for each of the elements of oral function, the estimate value calculated for each of the elements of oral function. The oral function evaluation indicator is an indicator for evaluating oral function. For example, the oral function evaluation indicator is a condition for assessing that oral function has deteriorated. The oral function evaluation indicator will be described with reference to FIG. 11.

FIG. 11 is a diagram illustrating an example of oral function evaluation indicators.

An oral function evaluation indicator is determined for each of the elements of oral function. For example, an indicator of 50% or more is determined for tongue fur adhesion, an indicator of 27 or less is determined for oral mucous wetness, an indicator of less than 500 N is determined for occlusal force (when DENTAL PRESCALE II from GC Corporation is used), an indicator of less than 30 kPa is determined for tongue pressure, and an indicator of less than 100 mg/dL is determined for mastication function (for the indicators, see “Koukukinouteikashou ni kansuru kihonteki na kangaekata (in Japanese) (Basic approaches to oral hypofunction) (https://www.jads.jp/basic/pdf/document_02.pdf)” in Japanese Association for Dental Science). Evaluator 140 evaluates a deterioration state of oral function of evaluatee U for each of the elements of oral function by comparing the estimate value calculated for each of the elements of oral function with the oral function evaluation indicator determined for each of the elements of oral function. For example, when the estimate value of tongue fur adhesion calculated is 50% or more, oral hygiene as an element of oral function is evaluated as being in a deteriorated state. Likewise, when the estimate value of oral mucous wetness calculated is 27 or less, oral mucous wetness as an element of oral function is evaluated as being in a deteriorated state; when the estimate value of occlusal force calculated is less than 500 N, occlusal force as an element of oral function is evaluated as being in a deteriorated state; when the estimate value of tongue pressure calculated is less than 30 kPa, tongue pressure as an element of oral function is evaluated as being in a deteriorated state; and when the estimate value of mastication function calculated is less than 100 mg/dL, mastication function as an element of oral function is evaluated as being in a deteriorated state. It should be noted that those shown in FIG. 11 as the oral function evaluation indicators determined for tongue fur adhesion, oral mucous wetness, occlusal force, tongue pressure, and mastication function are mere examples, and the oral function evaluation indicators are not limited to these. For example, an indicator for the remaining number of teeth may be determined for mastication function. Furthermore, tongue fur adhesion, oral mucous wetness, occlusal force, tongue pressure, and mastication function are shown as elements of oral function, but are mere examples. For example, for tongue-lip motor hypofunction, elements such as tongue movement, lip movement, and lip strength are applicable as elements of oral function.

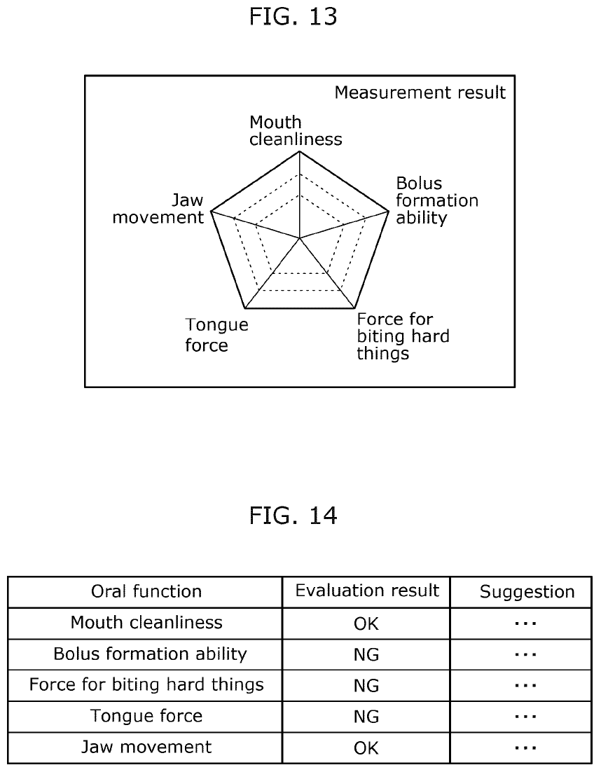

Returning to the description of FIG. 3A, outputter 150 outputs an evaluation result on oral function of evaluatee U evaluated by evaluator 140 (step S106). For example, outputter 150 outputs the evaluation result to mobile terminal 300. In this case, for example, outputter 150 may include a communication interface that performs wired communication or wireless communication. Outputter 150 obtains from storage 170 image data on an image corresponding to the evaluation result and transmits the obtained image data to mobile terminal 300. An example of the image data (evaluation result) is illustrated in FIG. 12 and FIG. 13.

FIG. 12 is a table and FIG. 13 is a chart each showing an example of the evaluation results on the elements of oral function. As shown in FIG. 12, each evaluation result may indicate one of two levels: OK or NG. OK means being normal, and NG means being abnormal. It should be noted that normal or abnormal need not be indicated for each element of oral function. For example, only an evaluation result of an element that is suspected of deteriorating may be indicated. Furthermore, the evaluation result is not limited to two levels, and may be in three or more fractionalized levels of evaluation. In this case, indicator data 172 stored in storage 170 may include a plurality of indicators for one element. Alternatively, as shown in FIG. 13, the evaluation result may be expressed in a radar chart. FIG. 12 and FIG. 13 show, as elements of oral function, mouth cleanliness, bolus formation ability, force for biting hard things, tongue force, and jaw movement. The evaluation result is presented based on the estimate value of tongue fur adhesion for mouth cleanliness, the estimate value of oral mucous wetness for bolus formation ability, the estimate value of occlusal force for force for biting hard things, the estimate value of tongue pressure for tongue force, and the estimate value of mastication function for jaw movement. It should be noted that FIG. 12 and FIG. 13 are mere examples, and wording which describes the evaluation items, items of oral function, and combinations of such corresponding wording and items are not limited to those in FIG. 12 and FIG. 13.

Returning to the description of FIG. 3A, suggester 160 provides a suggestion regarding oral function of evaluatee U by checking the estimate value calculated by calculator 130 against predetermined data (suggestion data 173) (step S107). Here, the predetermined data will be described with reference to FIG. 14.

FIG. 14 is an example of predetermined data (suggestion data 173) that is used when providing a suggestion regarding oral function.

As shown in FIG. 14, suggestion data 173 is data in which an evaluation result and details of a suggestion are associated with each other for each of the elements of oral function. For example, when the estimate value of mouth cleanliness calculated is less than 50%, the indicator is satisfied. Therefore, suggester 160 determines mouth cleanliness as OK and provides a suggestion based on details of suggestion associated with mouth cleanliness. It should be noted that although descriptions of specific details of suggestions are omitted, storage 170 stores data indicating details of suggestions (e.g., image, video, voice, text, etc.), and suggester 160 provides a suggestion regarding oral function to evaluatee U using such data, for example.

### Advantageous Effects Etc.

As described above, the voice feature calculation method according to a first aspect of the present disclosure is a voice feature calculation method, performed by a computer, for calculating one or more prosody features (features) of a voice of evaluatee U from a voice uttered by evaluatee U, the voice feature calculation method including: obtaining voice data obtained by collecting a voice uttered by evaluatee U; adjusting a sound pressure of the voice data obtained, based on a first average intensity of a sound that is included in the voice data obtained and is collected in a period in which evaluatee U does not utter a voice; and calculating, from the voice data resulting from the adjusting of the sound pressure, the one or more prosody features including at least a feature related to a sound pressure.

Accordingly, when a feature related to a sound pressure calculated from the voice uttered by evaluatee U is unsuitable to be used as-is for the evaluation of oral function, for example, a prosody feature with an appropriate feature related to a sound pressure can be calculated from the voice data resulting from the sound pressure adjustment. Accordingly, the feature related to a sound pressure calculated becomes more appropriate in terms of use for the evaluation of oral function of evaluatee U, for example. That is to say, it is possible to more appropriately calculate a prosody feature of a voice from a voice of the evaluatee.

Also, for example, a voice feature calculation method according to a second aspect may be the voice feature calculation method according to the first aspect, further including: calculating a signal-to-noise (S/N) ratio that is a ratio of a second average intensity to the first average intensity, the second average intensity being an average intensity of a sound that is included in the voice data obtained and is collected in a period in which evaluatee U utters a voice, wherein the sound pressure of the voice data may be adjusted when the S/N ratio calculated is less than or equal to a first threshold.

Accordingly, based on the condition of whether or not the S/N ratio is less than or equal to the first threshold, it is possible to determine whether the feature related to a sound pressure is unsuitable to be used as-is. According to the result of the determination, the sound pressure of the voice data can be adjusted and the feature related to a sound pressure can be calculated from the voice data resulting from the sound pressure adjustment. Accordingly, the feature related to a sound pressure calculated becomes more appropriate in terms of use for the evaluation of oral function of evaluatee U, for example. That is to say, it is possible to more appropriately calculate a prosody feature of a voice from a voice of the evaluatee.

Also, for example, a voice feature calculation method according to a third aspect may the voice feature calculation method according to the second aspect, wherein the sound pressure of the voice data may be a sound pressure not adjusted when the S/N ratio calculated is greater than the first threshold.

Accordingly, based on the condition of whether or not the S/N ratio is greater than the first threshold, it is possible to determine whether the feature related to a sound pressure is suitable to be used as-is. According to the result of the determination, the sound pressure of the voice data is not adjusted, and a feature related to a sound pressure can be calculated from the voice data the sound pressure of which is unchanged. Accordingly, the feature related to a sound pressure calculated becomes more appropriate in terms of use for the evaluation of oral function of evaluatee U, for example. That is to say, it is possible to more appropriately calculate a prosody feature of a voice from a voice of the evaluatee.

Also, for example, a voice feature calculation method according to a fourth aspect may the voice feature calculation method according to any one of the first through third aspects, wherein in the adjusting of the sound pressure of the voice data, the sound pressure may be adjusted by subtracting, from the sound pressure of the voice data, a sound pressure equivalent to a difference between a premeasured intensity of a sound during quiet and the first average intensity.

Accordingly, by (i) obtaining the sound pressure equivalent to the difference between: a premeasured intensity of a sound during quiet that corresponds to the proper sound pressure during quiet; and the intensity of a sound collected when evaluatee U does not utter a voice under the condition of sound collection in which the voice in the voice data was collected, and then (ii) subtracting the difference, the sound pressure can be adjusted so that the intensity of a sound collected when evaluatee U does not utter a voice becomes the intensity corresponding to the proper sound pressure during quiet.

Also, for example, a voice feature calculation method according to a fifth aspect may be the voice feature calculation method according to any one of the first through fourth aspects, wherein the adjusting of the sound pressure of the voice data may be performed at timing when the sound pressure indicates a local minimum value in the voice data.

Accordingly, the time when evaluatee U does not utter a voice can be identified based on the timing when the sound pressure indicates a local minimum value, and the sound pressure can be adjusted at the identified time.

Also, for example, a voice feature calculation method according to a sixth aspect may be the voice feature calculation method according to the fifth aspect, wherein the adjusting of the sound pressure of the voice data may be performed at timing when a fundamental frequency indicates zero in the voice data.

Accordingly, the time when evaluatee U does not utter a voice can be identified based on the timing when the fundamental frequency indicates zero in the voice data, and the sound pressure can be adjusted at the identified time.

Also, for example, a voice feature calculation method according to a seventh aspect may be the voice feature calculation method according to any one of the first through sixth aspects, wherein the one or more features may include: at least one of a sound pressure difference or a change in a sound pressure difference; and at least one of a formant, a change in a formant, a time length with mouth opened, a time length with mouth closed, a time length of a plosive, or a speech rate.

Accordingly, the calculated one or more features can include (i) at least one of a sound pressure difference or a change in a sound pressure difference as a feature related to a sound pressure and (ii) at least one of a formant, a change in a formant, a time length with mouth opened, a time length with mouth closed, a time length of a plosive, or a speech rate as another feature.

Also, for example, a voice feature calculation method according to an eighth aspect may be the voice feature calculation method according to the second or third aspect, further including: outputting information for increasing the S/N ratio when the S/N ratio calculated is less than or equal to a second threshold.

Accordingly, when a prosody feature is to be calculated from voice data collected in an unsuitable environment where the S/N ratio is less than the second threshold that is less than the first threshold, improvement of the environment can be prompted. It is possible to inhibit calculation of a prosody feature from voice data collected in such an unsuitable environment.

Also, for example, a voice feature calculation method according to a ninth aspect may be the voice feature calculation method according to the second or third aspect, further including: outputting information for increasing the S/N ratio when the first average intensity is greater than or equal to a sound pressure threshold.

Accordingly, when a prosody feature is to be calculated from voice data collected in an unsuitable environment where the first average intensity is greater than or equal to the sound pressure threshold, improvement of the environment can be prompted. It is possible to inhibit calculation of a prosody feature from voice data collected in such an unsuitable environment.

Furthermore, for example, as shown in FIG. 3A, the voice feature calculation method may include: obtaining voice data obtained by collecting a voice of evaluatee U uttering a syllable or a fixed sentence that includes (i) two or more morae including a change in a first formant frequency or a change in a second formant frequency or (ii) at least one of a flap, a plosive, a voiceless sound, a double consonant, or a fricative (step S102); extracting a prosody feature from the voice data obtained (step S103); calculating an estimate value of oral function of evaluatee U, based on the prosody feature extracted and an oral function estimating equation calculated based on a plurality of training data items (step S104); and evaluating a deterioration state of the oral function of evaluatee U by assessing the estimate value using an oral function evaluation indicator (step S105).

Accordingly, obtaining voice data suitable for evaluation of oral function makes it possible to evaluate oral function of evaluatee U in a simple and easy manner. In other words, simply by evaluatee U uttering the syllable or fixed sentence toward a sound collection device such as mobile terminal 300, it is possible to evaluate oral function of evaluatee U. In particular, since an estimate value of oral function is calculated using an estimating equation calculated based on a plurality of training data items, a deterioration state of oral function can be evaluated quantitatively. Furthermore, oral function is not evaluated by comparing a prosody feature directly with a threshold; rather, an estimate value is calculated from a prosody feature and an estimating equation, and the estimate value is compared with a threshold (oral function evaluation indicator). Therefore, a deterioration state of oral function can be evaluated with high precision.

For example, the estimating equation may include a coefficient corresponding to an element of oral function and a variable that is substituted by the prosody feature extracted and is multiplied by the coefficient.

Accordingly, an estimate value of oral function can be easily calculated, simply by substituting the extracted prosody feature into the estimating equation.

For example, in the calculating, the estimate value may be calculated for each of elements of oral function of evaluatee U, and in the evaluating, a deterioration state of oral function of evaluatee U may be evaluated for each of the elements of oral function by assessing, using an oral function evaluation indicator determined for each of the elements of oral function, the estimate value calculated for each of the elements of oral function.

Accordingly, the deterioration state of oral function can be evaluated for each element. For example, by preparing, for the respective elements of oral function, estimating equations including coefficients that differ according to the elements of oral function, it is possible to easily evaluate the deterioration state of oral function for each element.

For example, the elements of oral function may include at least one of tongue fur adhesion, oral mucous wetness, occlusal force, tongue pressure, cheek pressure, the remaining number of teeth, swallowing function, or mastication function of evaluatee U.

Accordingly, it is possible to evaluate a deterioration state regarding at least one of the following elements of oral function of evaluatee U: tongue fur adhesion, oral mucous wetness, occlusal force, tongue pressure, cheek pressure, the remaining number of teeth, swallowing function, or mastication function.

For example, the prosody feature may include at least one of a speech rate, a sound pressure difference, a change over time in the sound pressure difference, the first formant frequency, the second formant frequency, an amount of change in the first formant frequency, an amount of change in the second formant frequency, a change over time in the first formant frequency, a change over time in the second formant frequency, or a time length of a plosive.

Deterioration in oral function causes a change in pronunciation. Therefore, the deterioration state of oral function can be evaluated from these prosody features.

For example, in the extracting, a plurality of prosody features may be extracted from voice data obtained by collecting a voice of evaluatee U uttering two or more types of syllables or two or more types of fixed sentences, and in the calculating, an estimate value may be calculated based on the plurality of prosody features extracted and the estimating equation.

Accordingly, by using, for one estimating equation, the plurality of prosody features extracted based on two or more types of syllables or two or more types of fixed sentences, the precision of the calculation of an estimate value of oral function can be increased.

For example, the syllable or the fixed sentence may include a combination of two or more vowels or a vowel and a consonant. Here, the combination involves mouth opening and closing or back and forth tongue movement for utterance.

Accordingly, a prosody feature including an amount of change in the first formant frequency, a change over time in the first formant frequency, an amount of change in the second formant frequency, or a change over time in the second formant frequency can be extracted from a voice of evaluatee U uttering such a syllable or fixed sentence.

For example, the voice data may be obtained by collecting a voice of evaluatee U uttering a syllable or a fixed sentence at least twice at different speech rates.

Accordingly, the maintenance level of the state of oral function can be estimated from a voice of evaluatee U uttering such a syllable or fixed sentence.

For example, the fixed sentence may include repetition of syllables including a flap and a consonant different from the flap.

Accordingly, prosody features including a change over time in sound pressure difference, a change over time in sound pressure, and the number of repetitions can be extracted from a voice of evaluatee U uttering such syllables or a fixed sentence.

For example, the syllable or fixed sentence may include at least one combination of a vowel and a plosive.

Accordingly, prosody features including a sound pressure difference and a time length of a plosive can be extracted from a voice of evaluatee U uttering such a syllable or fixed sentence.

For example, the oral function evaluation method may further include providing a suggestion regarding oral function of evaluatee U by checking the estimate value against predetermined data.

Accordingly, evaluatee U can receive a suggestion on what measures should be taken when the oral function deteriorates.

Voice feature calculation device 400 according to a tenth aspect of the present disclosure is voice feature calculation device 400 that calculates one or more features of a voice of evaluatee U from a voice uttered by evaluatee U and includes: obtainer 110 that obtains voice data obtained by collecting a voice uttered by evaluatee U; sound pressure adjuster 116 that adjusts a sound pressure of the voice data obtained, based on a first average intensity of a sound that is included in the voice data and is collected in a period in which evaluatee U does not utter a voice; and extractor 120 that calculates the one or more features including at least a feature related to a sound pressure, by extracting the one or more features including at least the feature related to a sound pressure, from the voice data resulting from the adjustment of the sound pressure.

Accordingly, it is possible to achieve the same advantageous effects as those achieved by the voice feature calculation method described above.

Oral function evaluation device 100 according to an eleventh aspect of the present disclosure includes: voice feature calculation device 400 according to the tenth aspect; calculator 130 that calculates an estimate value of oral function of evaluatee U, based on: an estimating equation including the feature related to a sound pressure among the one or more features extracted from the voice data; and the one or more features extracted from the voice data resulting from the adjustment of the sound pressure; and evaluator 140 that evaluates a deterioration state of the oral function of evaluatee U by assessing the estimate value using an oral function evaluation indicator.

Accordingly, oral function of evaluatee U can be evaluated using voice feature calculation device 400.

For example, oral function evaluation device 100 may further include: a sound collection device (microphone) used for collecting a voice uttered by evaluatee U; and a presentation device (mobile terminal 300) that presents the deterioration state of the oral function of evaluatee U evaluated.

Furthermore, for example, oral function evaluation device 100 may include: obtainer 110 that obtains voice data obtained by collecting a voice of evaluatee U uttering a syllable or a fixed sentence that includes (i) two or more morae including a change in a first formant frequency or a change in a second formant frequency or (ii) at least one of a flap, a plosive, a voiceless sound, a double consonant, or a fricative; extractor 120 that extracts a prosody feature from the voice data obtained; calculator 130 that calculates an estimate value of oral function of evaluatee U, based on the prosody feature extracted and an oral function estimating equation calculated based on a plurality of training data items; and evaluator 140 that evaluates a deterioration state of oral function of evaluatee U by assessing the estimate value using an oral function evaluation indicator.

Accordingly, it is possible to provide oral function evaluation device 100 capable of evaluating oral function of evaluatee U in a simple and easy manner.

Furthermore, oral function evaluation system 200 may include, for example, oral function evaluation device 100 and a sound collection device (mobile terminal 300) that collects in a contactless manner a voice of evaluatee U uttering a syllable or a fixed sentence.

Accordingly, it is possible to provide oral function evaluation system 200 capable of evaluating oral function of evaluatee U in a simple and easy manner.

### Other Embodiments

The oral function evaluation method and so on according to the present embodiment have been described above, but the present invention is not limited to the above embodiment.

For example, the candidate estimating equations may be updated based on an evaluation result obtained by a specialist actually diagnosing oral function of evaluatee U. Accordingly, precision of the evaluation of oral function can be increased. Machine learning may be used to increase the precision of the evaluation of oral function.

For example, the details of suggestion may be evaluated by evaluatee U, and suggestion data 173 may be updated based on the evaluation result. For example, in the case where a suggestion is provided regarding oral function that is unproblematic for evaluatee U, evaluatee U evaluates the details of the suggestion as wrong. By updating suggestion data 173 based on this evaluation result, a wrong suggestion such as the one above is inhibited from being provided. This way, the details of a suggestion regarding oral function for evaluatee U can be made more effective. It should be noted that machine learning may be used to make the details of a suggestion regarding oral function more effective.

For example, evaluation results on oral function may be accumulated together with personal information items as big data, and the big data may be used for machine learning. Furthermore, the details of suggestions regarding oral function may be accumulated together with personal information items as big data, and the big data may be used for machine learning.

Further, for example, although the oral function evaluation method in the above embodiment includes providing a suggestion regarding oral function (step S107), this process need not be included. In other words, oral function evaluation device 100 need not include suggester 160.

Further, for example, although the personal information on evaluatee U is obtained in the obtaining of voice data (step S102) in the above embodiment, the personal information on evaluatee U need not be obtained. In other words, obtainer 110 need not obtain the personal information on evaluatee U.

Furthermore, for example, the steps included in the oral function evaluation method may be executed by a computer (a computer system). The present invention can be implemented as a program for causing a computer to execute the steps included in the oral function evaluation method. In addition, the present invention can be implemented as a non-transitory computer-readable recording medium such as a CD-ROM having such a program recorded thereon.

For example, in the case where the present invention is implemented using a program (a software product), each step is performed as a result of the program being executed using hardware resources such as a CPU, memory, and an input and output circuit of a computer. That is to say, each step is performed by the CPU obtaining data from, for example, the memory or the input and output circuit and performing calculation on the data, and outputting the calculation result to the memory or the input and output circuit, for example.

Further, each of the constituent elements included in oral function evaluation device 100 and oral function evaluation system 200 according to the above embodiment may be implemented as a dedicated or general-purpose circuit.

Further, each of the constituent elements included in oral function evaluation device 100 and oral function evaluation system 200 according to the above embodiment may be implemented as a large-scale integrated (LSI) circuit, which is an integrated circuit (IC).

The integrated circuit is not limited to an LSI and may be implemented as a dedicated circuit or a general-purpose processor. A field programmable gate array (FPGA) that allows for programming, or a reconfigurable processor that allows for reconfiguration of the connection and the setting of circuit cells inside an LSI may be employed.

Furthermore, when advancement in semiconductor technology or derivatives of other technologies brings forth a circuit integration technology which replaces LSI, such a circuit integration technology may be used to integrate the constituent elements included in oral function evaluation device 100 and oral function evaluation system 200.

The present invention also includes other forms achieved by making various modifications to the embodiments that may be conceived by those skilled in the art, as well as forms implemented by arbitrarily combining the constituent elements and functions in each embodiment without materially departing from the essence of the present invention.

### REFERENCE SIGNS LIST

100 oral function evaluation device
110 obtainer
115 S/N ratio calculator
116 sound pressure adjuster
120 extractor
130 calculator
140 evaluator
150 outputter
160 suggester
180 information outputter
200 oral function evaluation system
300 mobile terminal (terminal, microphone, presentation device)
400 voice feature calculation device
U evaluatee

1. A voice feature calculation method, performed by a computer, for calculating one or more features of a voice of an evaluatee from a voice uttered by the evaluatee, the voice feature calculation method comprising:
obtaining voice data obtained by collecting a voice uttered by the evaluatee;
adjusting a sound pressure of the voice data obtained, based on a first average intensity of a sound that is included in the voice data obtained and is collected in a period in which the evaluatee does not utter a voice; and
calculating, from the voice data resulting from the adjusting of the sound pressure, the one or more features including at least a feature related to a sound pressure.

2. The voice feature calculation method according to claim 1, further comprising:
calculating a signal-to-noise (S/N) ratio that is a ratio of a second average intensity to the first average intensity, the second average intensity being an average intensity of a sound that is included in the voice data obtained and is collected in a period in which the evaluatee utters a voice, wherein
the sound pressure of the voice data is adjusted when the S/N ratio calculated is less than or equal to a first threshold.

3. The voice feature calculation method according to claim 2, wherein
the sound pressure of the voice data is not adjusted when the S/N ratio calculated is greater than the first threshold.

4. The voice feature calculation method according to claim 1, wherein
in the adjusting of the sound pressure of the voice data,
the sound pressure is adjusted by subtracting, from the sound pressure of the voice data, a sound pressure equivalent to a difference between a premeasured intensity of a sound during quiet and the first average intensity.

5. The voice feature calculation method according to claim 1, wherein
the adjusting of the sound pressure of the voice data is performed at timing when the sound pressure indicates a local minimum value in the voice data.

6. The voice feature calculation method according to claim 5, wherein
the adjusting of the sound pressure of the voice data is performed at timing when a fundamental frequency indicates zero in the voice data.

7. The voice feature calculation method according to claim 1, wherein
the one or more features include: at least one of a sound pressure difference or a change in a sound pressure difference; and at least one of a formant, a change in a formant, a time length with mouth opened, a time length with mouth closed, a time length of a plosive, or a speech rate.

8. The voice feature calculation method according to claim 2, further comprising:
outputting information for increasing the S/N ratio when the S/N ratio calculated is less than or equal to a second threshold.

9. The voice feature calculation method according to claim 2, further comprising:
outputting information for increasing the S/N ratio when the first average intensity is greater than or equal to a sound pressure threshold.

10. A voice feature calculation device that calculates one or more features of a voice of an evaluatee from a voice uttered by the evaluatee, the voice feature calculation device comprising:
an obtainer that obtains voice data obtained by collecting a voice uttered by the evaluatee;
a sound pressure adjuster that adjusts a sound pressure of the voice data obtained, based on a first average intensity of a sound that is included in the voice data and is collected in a period in which the evaluatee does not utter a voice; and
an extractor that calculates the one or more features including at least a feature related to a sound pressure, by extracting the one or more features including at least the feature related to a sound pressure, from the voice data resulting from the adjustment of the sound pressure.

11. An oral function evaluation device comprising:
the voice feature calculation device according to claim 10;
a calculator that calculates an estimate value of oral function of the evaluatee, based on: an estimating equation including the feature related to a sound pressure among the one or more features extracted from the voice data; and the one or more features extracted from the voice data resulting from the adjustment of the sound pressure; and
an evaluator that evaluates a deterioration state of the oral function of the evaluatee by assessing the estimate value using an oral function evaluation indicator.
